(19) United States
(12) Patent Application Publication
(10) Pub. No.: US 2026/0110693 A1
(43) Pub. Date: Apr. 23, 2026

(54) ASSAYS FOR LYSOSOMAL STORAGE DISEASES

(71) Applicant: SANGAMO THERAPEUTICS, INC., Richmond, CA (US)

(72) Inventors: Ruby CHEUNG, Richmond, CA (US); Joshua N. HOLTER, Richmond, CA (US); Liching C. CAO, Richmond, CA (US)

(21) Appl. No.: 19/120,513

(22) PCT Filed: Oct. 13, 2023
(86) PCT No.: PCT/US2023/076817

§ 371 Date: Apr. 11, 2025

Related U.S. Application Data
(60) Provisional application No. 63/379,597, filed on Oct. 14, 2022.

(51) Int. Cl. G01N 33/68; G01N 33/543; G01N 33/544

(52) U.S. Cl. CPC G01N 33/6854; G01N 33/54306; G01N 33/54393; G01N 33/544; G01N 33/6893; G01N 2800/04

(57) ABSTRACT

The disclosure provides the methods of detecting an anti-lysosomal enzyme antibody in a human subject (e.g., in the presence of a circulating lysosomal enzyme) and the methods of improving an efficacy of an anti-lysosomal enzyme antibody assay (e.g., improving the assay sensitivity and the circulating lysosomal enzyme tolerance), comprising pre-treating the biological sample of the subject with a base at pH of about 11 or greater and measuring the presence of the anti-lysosomal enzyme antibody in the biological sample of the subject.

### CROSS REFERENCE TO RELATED APPLICATIONS

This application claims the benefit of the filing date of U.S. Provisional Patent Application No. 63/379,597, filed Oct. 14, 2022, the contents of which are incorporated herein by reference in its entirety.

### FIELD OF DISCLOSURE

The present disclosure provides the methods of detecting an anti-lysosomal enzyme antibody in a human subject (e.g., in the presence of a circulating lysosomal enzyme) and the methods of improving an efficacy of an anti-lysosomal enzyme antibody assay (e.g., improving the assay sensitivity and the circulating lysosomal enzyme tolerance), comprising pre-treating the biological sample of the subject with a base at pH of about 11 or greater and measuring the presence of the anti-lysosomal enzyme antibody in the biological sample of the subject.

### BACKGROUND OF THE DISCLOSURE

There is significant immunogenicity towards current treatments for the lysosomal storage disorders (LSDs), and therefore it is important to be able to measure the anti-lysosomal enzyme antibodies in patients to understand how and if new drugs will work in these patients. For lysosomal storage disorders with currently approved enzyme replacement therapies (ERTs), there is a significant incidence of the anti-ERT antibodies including the anti-ERT neutralizing antibodies.

Current methods to accurately measure the anti-lysosomal enzyme antibodies can require blood draws at trough drug level to ensure no residual lysosomal enzyme is present in the blood, as residual enzyme can interfere with circulating antibodies by forming complexes which limit detection. For treatments such as gene therapy, it is not possible to withdraw treatment and dosing, and therefore the anti-lysosomal enzyme antibodies cannot be measured accurately.

Accordingly, there is a need for improving the methods of accurately measuring the anti-lysosomal enzyme antibodies and, thus, improving customizing treatments for patients diagnosed with a lysosomal storage disorder.

### SUMMARY OF THE DISCLOSURE

In some aspects, the disclosure is directed to a method of detecting an anti-lysosomal enzyme antibody in a human subject, comprising measuring the presence of the anti-lysosomal enzyme antibody in a biological sample of the subject, wherein the biological sample is pre-treated with a base at pH of about 11 or greater.

In some aspects, the methods of the disclosure comprise detecting the anti-lysosomal enzyme antibody in the presence of a circulating lysosomal enzyme.

In some aspects, the disclosure is directed to a method of improving an efficacy of an anti-lysosomal enzyme antibody assay, comprising pre-treating a biological sample of a human subject with a base at pH of about 11 or greater, further comprising measuring the presence of the anti-lysosomal enzyme antibody in the biological sample. In some aspects, improving an efficacy of the anti-lysosomal enzyme antibody assay of the disclosure comprises improving the assay sensitivity and the circulating lysosomal enzyme tolerance.

In some aspects, wherein the base is a non-buffered base. In some aspects, the non-buffered base is NaOH or Ca(OH)₂. In some aspects, NaOH or Ca(OH)₂ is about 0.01M, about 0.02M, about 0.03M, about 0.04M, about 0.05M, about 0.06M, about 0.07M, about 0.08M, about 0.09M, or about 0.1M. In some aspects, NaOH or Ca(OH)₂ is about 0.02M.

In some aspects, the biological sample is a serum sample or a plasma sample. In some aspects,

In some aspects, the biological sample is diluted at minimum required dilution (MRD) of about 2 fold or greater.

In some aspects, the biological sample is diluted at MRD of about 1 fold, 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 26 fold, about 27 fold, about 28 fold, about 29 fold, about 30 fold, about 31 fold, about 32 fold, about 33 fold, about 34 fold, about 35 fold, about 36 fold, about 37 fold, about 38 fold, about 39 fold, about 40 fold, about 41 fold, about 42 fold, about 43 fold, about 44 fold, about 45 fold, about 46 fold, about 47 fold, about 48 fold, about 49 fold, about 50 fold, about 51 fold, about 52 fold, about 53 fold, about 54 fold, about 55 fold, about 56 fold, about 57 fold, about 58 fold, about 59 fold, about 60 fold, about 61 fold, about 62 fold, about 63 fold, about 64 fold, about 65 fold, about 66 fold, about 67 fold, about 68 fold, about 69 fold, about 70 fold, about 71 fold, about 72 fold, about 73 fold, about 74 fold, about 75 fold, about 76 fold, about 77 fold, about 78 fold, about 79 fold, about 80 fold, about 81 fold, about 82 fold, about 83 fold, about 84 fold, about 85 fold, about 86 fold, about 87 fold, about 88 fold, about 89 fold, about 90 fold, about 91 fold, about 92 fold, about 93 fold, about 94 fold, about 95 fold, about 96 fold, about 97 fold, about 98 fold, about 99 fold, about 100 fold about 200 fold, about 300 fold, about 400 fold, about 500 fold, about 600 fold, about 700 fold, about 800 fold, about 900 fold, about 1000 fold, about 1100 fold, about 1200 fold, about 1300 fold, about 1400 fold, about 1500 fold, about 1600 fold, about 1700 fold, about 1800 fold, about 1900 fold, about 2000 fold, about 2100 fold, about 2200 fold, about 2300 fold, about 2400 fold, about 2500 fold, about 2600 fold, about 2700 fold, about 2800 fold, about 2900 fold, about 3000 fold, about 3100 fold, about 3200 fold, about 3300 fold, about 3400 fold, about 3500 fold, about 3600 fold, about 3700 fold, about 3800 fold, about 3900 fold, about 4000 fold, about 4100 fold, about 4200 fold, about 4300 fold, about 4400 fold, about 4500 fold, about 4600 fold, about 4700 fold, about 4800 fold, about 4900 fold, or about 5000 fold.

In some aspects, pH is about 11, about 11.1, about 11.15, about 11.2, about 11.25, about 11.3, about 11.35, about 11.4, about 11.45, about 11.5, about 11.55, about 11.6, about 11.65, about 11.7, about 11.75, about 11.8, about 11.85, about 11.9, about 11.95, about 12, 12.1, about 12.15, about 12.2, about 12.25, about 12.3, about 12.35, about 12.4, about 12.45, about 12.5, about 12.55, about 12.6, about 12.65, about 12.7, about 12.75, about 12.8, about 12.85, about 12.9, about 12.95, about 13, 13.1, about 13.15, about 13.2, about 13.25, about 13.3, about 13.35, about 13.4, about 13.45, about 13.5, about 13.55, about 13.6, about 13.65, about 13.7, about 13.75, about 13.8, about 13.85, about 13.9, about 13.95, or about 14.

In some aspects, pH is 12.45.

In some aspects, pH is greater than 11 and less than 12, greater than 11 and less than 13, or greater than 11 and less than 14.

In some aspects, the subject has a lysosomal storage disease. In some aspects, the lysosomal storage disease is selected from a group consisting of: Fabry disease, Gaucher disease, Pompe disease, Mucopolysaccharidoses (MPS) type I disease, MPS type II, MPS type III, MPS type IV, MPS type VI, MPS type VII, mucolipidosis (ML), Niemann-Pick disease, Tay-Sachs disease, and Batten disease.

In some aspects, the presence of an anti-lysosomal enzyme neutralizing antibody (NAb) is measured. In some aspects, the presence of anti-lysosomal enzyme total antibodies (TAbs) is measured.

In some aspects, the methods of the disclosure further comprise mixing the pre-treated biological sample with a lysosomal enzyme. In some aspects, the lysosomal enzyme is selected from a group consisting of: α-galactosidase A (a-Gal A), glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, and tripeptidly peptidase 1. In some aspects, the lysosomal enzyme is α-galactosidase A.

In some aspects, the neutralizing anti-lysosomal enzyme antibody is selected from a group consisting of: an anti-α-galactosidase A antibody, an anti-glucocerebrosidase antibody, an anti-alpha-glucosidase antibody, an anti-alpha-L-iduronidase antibody, an anti-iduronate 2-sulfatase antibody, an anti-sulfamidase antibody, an anti-galactosamine-6-sulfatase antibody, an anti-N-acetylgalactosamine-4 sulfatase antibody, an anti-beta-glucuronidase antibody, an anti-N-acetylglucosamine-1-phosphotransferase antibody, an anti-Niemann-Pick C1 protein antibody, an anti-hexosaminidase A, and an anti-tripeptidly peptidase 1 antibody. In some aspects, the neutralizing anti-lysosomal enzyme antibody is the anti-α-galactosidase A antibody.

In some aspects, the pre-treated biological sample and the lysosomal enzyme mixture is incubated for at least about one hour, at least about two hours, at least about three hours, at least about four hours, at least about five hours, at least about six hours, at least about seven hours, at least about eight hours, at least about nine hours, at least about ten hours, at least about eleven hours, at least about twelve hours, at least about thirteen hours, at least about fourteen hours, at least about fifteen hours, at least about sixteen hours, at least about seventeen hours, at least about eighteen hours, at least about nineteen hours, at least about twenty hours, at least about twenty one hours, at least about twenty two hours, at least about twenty three hours, or at least about twenty four hours.

In some aspects, the methods of the disclosure further comprise mixing the pre-treated biological sample and the lysosomal enzyme with a reaction mix.

In some aspects, the lysosomal enzyme is at a concentration of less than about 10 ug/mL, less than about 9 ug/mL, less than about 8 ug/mL, less than about 7 ug/mL, less than about 6 ug/mL, less than about 5 ug/mL, less than about 4 ug/mL, less than about 3 ug/mL, less than about 2 ug/mL, less than about 1 ug/mL, less than about 0.9 ug/mL, less than about 0.8 ug/mL, less than about 0.7 ug/mL, less than about 0.6 ug/mL, less than about 0.5 ug/mL, less than about 0.4 ug/mL, less than about 0.3 ug/mL, less than about 0.2 ug/mL, less than about 100 ng/ml, less than about 90 ng/ml, less than about 80 ng/ml, less than about 70 ng/ml, less than about 60 ng/ml, less than about 50 ng/ml, less than about 40 ng/ml, less than about 30 ng/ml, less than about 20 ng/ml, or less than about 10 ng/ml.

In some aspects, the pre-treated biological sample and the lysosomal enzyme mixture is incubated for at least about one hour, at least about two hours, at least about three hours, at least about four hours, at least about five hours, at least about six hours, at least about seven hours, at least about eight hours, at least about nine hours, at least about ten hours, at least about eleven hours, at least about twelve hours, at least about thirteen hours, at least about fourteen hours, at least about fifteen hours, at least about sixteen hours, at least about seventeen hours, at least about eighteen hours, at least about nineteen hours, at least about twenty hours, at least about twenty one hours, at least about twenty two hours, at least about twenty three hours, or at least about twenty four hours.

In some aspects, the pre-treated biological sample and the lysosomal enzyme mixture is incubated for a duration between about 1 and about 15 hours, between about 2 and about 14 hours, between about 3 and about 13 hours, between about 4 and about 12 hours, between about 5 and about 11 hours, between about 6 and about 10 hours, or between about 7 and about 9 hours.

In some aspects, the reaction mix comprises a substrate and/or an inhibitor. In some aspects, the substrate is selected from the group consisting of. 4-Methylumbelliferyl-α-D-galactopyranoside, 4-Methylumbelliferyl-β-D-glucopyranoside, 4-Methylumbelliferyl a-D-glucopyranoside, 4-Methylumbelliferyl alpha-L-idopyranoside, 4-Methylumbelliferyl-α-L-Iduronide 2-sulfate, 4-Methylumbellieryl-2-sulfamino-2-deoxy-α-D-glucopyranoside, 4-methylumbelliferyl-β-D-galactose-6-sulfate, 4-Methylumbellieryl-N-acetyl-α-d-galactoseaminide-4-sulfate, 4-methylumbelliferyl-β-d-glucuronide, 4-methylumbelliferyl-α-D-mannopyronoside, 4-Methylumbelliferyl N-acetyl-β-D-glucosaminide, and Ala-Ala-Phe-7-amido-4-methyl-coumarin.

In some aspects, the substrate is at a concentration of at least about 1.1 mM, at least about 1.2 mM, at least about 1.3 mM, at least about 1.4 mM, at least about 1.5 mM, at least about 1.6 mM, at least about 1.7 mM, at least about 1.8 mM, at least about 1.9 mM, at least about 2 mM, at least about 2.1 mM, at least about 2.2 mM, at least about 2.3 mM, at least about 2.4 mM, at least about 2.5 mM, at least about 2.6 mM, at least about 2.7 mM, at least about 2.8 mM, at least about 2.9 mM, at least about 3 mM, at least about 3.1 mM, at least about 3.2 mM, at least about 3.3 mM, at least about 3.4 mM, at least about 3.5 mM, at least about 3.6 mM, at least about 3.7 mM, at least about 3.8 mM, at least about 3.9 mM, at least about 4 mM, at least about 4.1 mM, at least about 4.2 mM, at least about 4.3 mM, at least about 4.4 mM, at least about 4.5 mM, at least about 4.6 mM, at least about 4.7 mM, at least about 4.8 mM, at least about 4.9 mM, or at least about 5 mM.

In some aspects, the inhibitor comprises N-Acetylgalactosamine (GALNAc).

In some aspects, the inhibitor is at a concentration of less than about 200 mM, less than about 195 mM, less than about 190 mM, less than about 185 mM, less than about 180 mM, less than about 175 mM, less than about 170 mM, less than about 165 mM, less than about 160 mM, less than about 155 mM, less than about 150 mM, less than about 145 mM, less than about 140 mM, less than about 135 mM, less than about 130 mM, less than about 125 mM, less than about 120 mM, less than about 115 mM, or less than about 110 mM.

In some aspects, the reaction mix and the pre-treated biological sample with the lysosomal enzyme mixture are combined in a high throughput plate.

In some aspects, the reaction mix and the pre-treated biological sample with the lysosomal enzyme mixture are incubated at room temperature at revolutions per minute (RPM) 300, 400, 500, or 600.

In some aspects, the methods of the disclosure further comprise adding a stop buffer to the mixture after incubation. In some aspects, the incubation period is at least about 30 minutes, at least about 35 minutes, at least about 40 minutes, at least about 45 minutes, at least about 50 minutes, at least about 55 minutes, at least about 60 minutes, at least about 65 minutes, at least about 70 minutes, at least about 75 minutes, or at least about 80 minutes.

In some aspects, the stop buffer comprises glycine. In some aspects, the stop buffer is at a volume of less than about 1 mL, less than about 900 uL, less than about 800 uL, less than about 700 uL, less than about 600 uL, less than about 500 uL, less than about 400 uL, less than about 300 uL, less than about 200 uL, or less than about 100 uL.

In some aspects, the methods of the disclosure further comprise neutralizing the pre-treated biological sample with an acid. In some aspects, the acid is acetic acid or hydrochloric acid. In some aspects, the acetic acid or hydrochloric acid is about 20 mM, about 30 mM, about 40 mM, about 50 mM, about 60 mM, about 70 mM, about 80 mM, about 90 mM, about 100 mM, about 200 mM, about 300 mM, about 400 mM, about 500 mM, or about 600 mM. In some aspects, the acetic acid is about 30 mM.

In some aspects, the methods of the disclosure further comprise mixing the neutralized pre-treated biological sample with a lysosomal enzyme.

In some aspects, the neutralized pre-treated biological sample and the lysosomal enzyme mixture is added to a high throughput plate coated with a lysosomal enzyme antigen. In some aspects, the lysosomal enzyme antigen is selected from a group consisting of: an a-Gal A antigen, a glucocerebrosidase antigen, an alpha-glucosidase antigen, an alpha-L-iduronidase antigen, a iduronate 2-sulfatase antigen, a sulfamidase antigen, a galactosamine-6-sulfatase antigen, a N-acetylgalactosamine-4 sulfatase antigen, a beta-glucuronidase antigen, a N-acetylglucosamine-1-phosphotransferase antigen, a Niemann-Pick C1 protein antigen, a hexosaminidase A antigen, and a tripeptidly peptidase 1 antigen. In some aspects, the lysosomal enzyme antigen is the a-Gal A antigen.

In some aspects, an enzyme-conjugated detection antibody or enzyme-conjugated lysosomal enzyme is added to the plate to produce a signal.

In some aspects, a detection antibody or lysosomal enzyme which has a ruthenylated label is added to the plate to produce a signal.

In some aspects, the detection antibody is an anti-human IgG antibody.

In some aspects, the methods of the disclosure further comprise adding a substrate. In some aspects, the substrate is converted by the enzyme on the detection antibody, producing a color reaction product. In some aspects, the plate is read in a plate reader which detects the color reaction product and outputs optical density (OD) values. In some aspects, the OD values represent the total levels of the anti-lysosomal neutralizing antibody.

In some aspects, the plate is read in a plate reader which detects light emissions by the ruthenylated label and outputs electrochemiluminescent units (ECLu). In some aspects, the ECLu represent the total levels of the anti-lysosomal total antibodies.

### BRIEF DESCRIPTION OF THE DRAWINGS

FIG. 1 shows the anti-enzyme replacement therapy (ERT) antibodies incidence including the including the anti-ERT neutralizing antibodies for the lysosomal storage diseases (Fabry disease, Pompe disease, Gaucher disease, and Mucopolysaccharidoses (MPS) type I disease, MPS type II disease, MPS type IV disease, and MPS type VI disease). The antibody incidence values were obtained from the approved drug label.

FIG. 2 shows the lysosomal enzyme neutralizing (NAb) antibody assay format.

FIG. 3A-3B show the effect of circulating levels of the lysosomal enzyme (on-board lysosomal enzyme concentration (ng/ml) [0 to 2000 ng/ml (FIG. 3A)] and [0 ng/ml to 500 ng/ml (FIG. 3B)] on the enzymatic neutralizing ability (% inhibition of the lysosomal enzyme activity (signal inhibition)) of polyclonal RP-01 antibody (the neutralizing anti-lysosomal enzyme antibody) at different concentrations (50 ug/ml, 100 ug/ml, and 150 ug/ml).

FIG. 4A-4B show the effect of heat pre-treatment of the biological sample on the enzymatic neutralizing ability (% inhibition of the lysosomal enzyme activity (signal inhibition)) of RP-01 antibody at different concentrations (0 ug, 50 ug, 100 ug, and 150 ug). FIG. 4A shows the results with no sample pre-treatment and FIG. 4B shows the results with 56° C. pre-treatment of the serum sample.

FIG. 5 shows various pre-treatment methods pH 11, Melon Gel, pH 2, and the standard assay diluent (AD) condition) assessed to test the ability to detect the anti-lysosomal enzyme neutralizing antibodies NAbs (RP-01) in the presence of the on-board lysosomal enzyme (concentration (ng/ml)).

FIG. 6 shows the effect of an alkaline (base) pre-treatment of the biological sample on the ability to detect the anti-lysosomal enzyme neutralizing antibodies NAbs (RP-01) at different concentrations (50 ug/ml, 100 ug/ml, and 150 ug/ml) in the presence of the on-board lysosomal enzyme (concentration (ng/ml)).

FIG. 7 shows the effect of an alkaline (base) pre-treatment of the biological sample on the ability to detect the anti-lysosomal enzyme neutralizing antibodies NAbs (monoclonal positive control anti-lysosomal enzyme NAb, 7H11) at different concentrations (125 ng/ml, 250 ng/ml, and 1000 ng/ml) in the presence of the on-board lysosomal enzyme (concentration (ng/ml)).

FIG. 8 shows the effect of a high molarity base pre-treatment of the biological sample on the ability to detect the anti-lysosomal enzyme neutralizing antibodies NAbs (monoclonal positive control anti-lysosomal enzyme NAb (7H11)) at different concentrations (50 ug/ml, 100 ug/ml, and 150 ug/ml) in the presence of the on-board lysosomal enzyme (concentration (ng/ml)).

FIG. 9 shows the ability to detect the anti-lysosomal enzyme neutralizing antibodies NAbs: polyclonal positive control anti-lysosomal enzyme NAb, (RP-01) at different concentrations (50 ug/ml, 100 ug/ml, and 150 ug/ml) and monoclonal positive control anti-lysosomal enzyme NAbs, (7H11 and 19D6) at different concentrations (125 ng/ml, 250 ng/ml, and 1000 ng/ml) in the presence of the on-board lysosomal enzyme (concentration (ng/ml)). as measured by the standard anti-lysosomal neutralizing antibody assay.

FIG. 10 shows the effect of an alkaline (base) pre-treatment of the biological sample on the ability to detect the anti-lysosomal enzyme neutralizing antibodies NAbs: polyclonal positive control anti-lysosomal enzyme NAb, (RP-01) at different concentrations (50 ug/ml, 100 ug/ml, and 150 ug/ml) and monoclonal positive control anti-lysosomal enzyme NAbs, (7H11 and 19D6) at different concentrations (125 ng/ml, 250 ng/ml, and 1000 ng/ml) in the presence of the on-board lysosomal enzyme (concentration (ng/ml))

FIG. 11 shows the lysosomal enzyme total antibody (TAb) assay format.

FIG. 12A-12B show the effect of various acidic pre-treatments (with or without heat pre-treatment (FIG. 12A) or with various base neutralization treatments (FIG. 12B)) on the ability to detect serum anti-lysosomal antibodies (as measured by 450 nm optical density (OD)) in the presence of the on-board lysosomal enzyme. Tested samples were 1 ug/ml 19D6 Ab+2 ug/ml α-Gal A; 1 ug/ml 19D6 Ab; and 2 ug/ml α-Gal A.

FIG. 13 shows the effect of various alkaline pre-treatments (pH 12, pH 11+heat, and pH 12+heat) on the ability to detect serum anti-lysosomal antibodies (as measured by 450 nm optical density (OD)) in the presence of the on-board lysosomal enzyme. Tested samples were 1 ug/ml 19D6 Ab+2 ug/ml α-Gal A; 1 ug/ml 19D6 Ab; and 2 ug/ml α-Gal A.

FIG. 14A-14B show the effect of neutral pH and basic pH pre-treatment on the ability to detect serum anti-lysosomal antibodies (as measured by 450 nm optical density (OD)) in the absence (FIG. 14A) or presence (FIG. 14B) of the on-board lysosomal enzyme.

FIG. 15 shows the measurement of the total anti-lysosomal enzyme antibodies (as measured by 450 nm optical density (OD)) in the serum sample after no treatment or alkaline pre-treatment of serum sample from an individual with no Fabry disease (negative serum); serum sample from individual #3 with Fabry disease in the presence of 4 ug/ml of on-board lysosomal enzyme (Fabry serum #3+4 ug/ml of α-Gal A); serum sample from individual #3 with Fabry disease (Fabry serum #3); and serum sample from individual #2 with Fabry disease (Fabry serum #2).

FIG. 16A-16B shows the measurement of the total anti-lysosomal enzyme antibodies (as measured by 450 nm optical density (OD)) in the serum sample after no treatment or alkaline pre-treatment of serum sample from individual #1 with Fabry disease in the absence or presence of on-board lysosomal enzyme (Fabry serum #1 and Fabry serum #1+α-Gal A, respectively) (FIG. 16A) and serum sample from individual #4 with Fabry disease in the absence or presence of on-board lysosomal enzyme (Fabry serum #4 and Fabry serum #4+α-Gal A, respectively) (FIG. 16B).

### DETAILED DESCRIPTION OF THE DISCLOSURE

The present disclosure provides the methods of detecting an anti-lysosomal enzyme antibody in a human subject (e.g., in the presence of a circulating lysosomal enzyme) and the methods of improving an efficacy of an anti-lysosomal enzyme antibody assay (e.g., improving the assay sensitivity and the circulating lysosomal enzyme tolerance), comprising pre-treating the biological sample of the subject with a base at pH of about 11 or greater and measuring the presence of the anti-lysosomal enzyme antibody in the biological sample of the subject.

### I. Terms

In order that the present disclosure can be more readily understood, some terms are first defined. As used in this application, except as otherwise expressly provided herein, each of the following terms shall have the meaning set forth below. Additional definitions are set forth throughout the application.

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides.

The term “lysosomal enzyme” refers to protein with enzymatic activity, such as for example, glycosidases, proteases, and sulfatases. Mammalian lysosomal enzymes are synthesized in the cytosol and traverse the endoplasmic reticulum (ER) where they are glycosylated with N-linked, high mannose type carbohydrate. In the Golgi apparatus, the high mannose carbohydrate is modified on lysosomal proteins by the addition of mannose-6-phosphate (M6P) which targets these proteins to the lysosome. The M6P-modified proteins are delivered to the lysosome via interaction with either of two M6P receptors. In some aspects, the lysosomal enzyme includes but is not limited to α-galactosidase A (α-Gal A), glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick Cl protein, hexosaminidase A, and tripeptidly peptidase 1.

The terms “α-Galactosidase A,” “α-Gal A,” and “GAL” are used interchangeably and refer to a protein with enzymatic activity comprising hydrolysis of terminal, non-reducing α-D-galactose residues in α-D-galactosides, including galactose oligosaccharides, galactomannans and galactolipids. In some aspects, α-Gal A comprises the enzyme described by IUBMB Enzyme Nomenclature EC 3.2.1.22 (as described, for example, in Suzuki et al., J. Biol. Chem. 245:781-786(1970); Wiederschain, G. and Beyer, E. Dokl. Akad. Nauk S.S.S.R. 231:486-488 (1976)). In some aspects, α-Gal A comprises a protein encoded by a nucleic acid comprising human GLA gene, for example, human α-Gal A gene defined by GenBank Accession No. NM_000169. In some aspects, α-Gal A comprises a protein comprising the amino acid sequence defined by GenBank Accession No. NP_000160. In some aspects, the α-Gal A is agalsidase alpha, produced by genetic engineering technology in a human cell line. Agalsidase alpha is available as Replagal®, from Shire Plc. (Dublin, Ireland). In some aspects, the α-Gal A is agalsidase beta, produced by recombinant DNA technology in a Chinese hamster ovary (CHO) cell line. Agalsidase beta is available as Fabrazyme®, from Sanofi Genzyme (Cambridge, Mass.). In some aspects, the α-Gal A is a recombinant human α-Gal A produced in CHO cells transformed with an expression vector encoding human α-Gal A gene (JCR Pharmaceuticals Co. Ltd, (Japan)), identified as JR-051.

The terms “glucocerebrosidase,” “GCase,” “β-Glucocerebrosidase,” “β-glucosidase,” “beta-Glucocerebrosidase,” “beta-glucosidase,” “D-glucosyl-N-acylsphingosine glucohydrolase,” “glucosylceramidase,” and “glucosylceramidase beta 1 (GBA1)” are used interchangeably and refer to an enzyme with glucosylceramidase activity that is needed to cleave, by hydrolysis, the beta-glycosidic linkage of the chemical glucocerebroside, an intermediate in glycolipid metabolism that is abundant in cell membranes (particularly skin cells). Glucocerebrosidase is localized in the lysosome, where it remains associated with the lysosomal membrane. (Rijnboutt et al., J. Biol. Chem. 266 (8): 4862-8 (1991)). Glucocerebrosidase is 497 amino acids in length and has a molecular weight of 59,700 Daltons. In some aspects, glucocerebrosidase comprises the enzyme described by IUBMB Enzyme Nomenclature EC 3.2.1.45 (as described, for example, in Boer et al., J. Clin. Med. 9(3):736 (2020)). In some aspects, glucocerebrosidase comprises a protein encoded by a nucleic acid comprising human glucocerebrosidase gene, for example, human glucocerebrosidase gene defined by GenBank Accession No. NM_000157, NM_001005741, or NM_001005742. In some aspects, glucocerebrosidase comprises a protein comprising the amino acid sequence defined by GenBank Accession No. NP_000148, NP_001005741, NP_001005742, NP_001165282, or NP_001165283.

The terms “alpha-glucosidase,” “α-glucosidase,” “acid alpha-glucosidase,” “GAA,” “lysosomal alpha-glucosidase,” “maltase,” “glucoinvertase,” “glucosidosucrase,” “maltase-glucoamylase,” “α-glucopyranosidase,” “glucosidoinvertase,” “α-D-glucosidase,” “α-glucoside hydrolase,” “α-1,4-glucosidase,” and “α-D-glucoside glucohydrolase” are used interchangeably and refer to an enzyme which is essential for the degradation of glycogen to glucose in lysosomes. Alpha-glucosidase has the highest activity on alpha-1,4-linked glycosidic linkages, but can also hydrolyze alpha-1,6-linked glucans. In some aspects, alpha-glucosidase comprises the enzyme described by IUBMB Enzyme Nomenclature EC 3.2.1.20 (as described, for example, in Bruni et al., Biochim. Biophys. Acta 212:470-477 (1970)). In some aspects, alpha-glucosidase comprises a protein encoded by a nucleic acid comprising human alpha-glucosidase gene, for example, human alpha-glucosidase gene defined by GenBank Accession No. NM_000152, NM_001079803, or NM_001079804. In some aspects, alpha-glucosidase comprises a protein comprising the amino acid sequence defined by GenBank Accession No. NP_000143, NP 001073271, or NP_001073272.

The terms “alpha-L-iduronidase,” “L-iduronidase,” “laronidase,” “α-L-iduronidase,” and “glycosaminoglycan alpha-L-iduronohydrolase” are used interchangeably and refer to an enzyme which is involved in the degeneration of glycosaminoglycans such as dermatan sulfate and heparan sulfate. The enzyme acts by hydrolyzing the terminal alpha-L-iduronic acid residues of these molecules, degrading them. Alpha-L-iduronidase is a glycoprotein enzyme found in the lysosomes of cells and is reported as having a mass of approximately 83 kilodaltons. In some aspects, alpha-L-iduronidase comprises the enzyme described by IUBMB Enzyme Nomenclature EC 3.2.1.76 (as described, for example, in Rome et al., Arch Biochem Biophys 189:344-53 (1978); Scott et al. Proc Natl Acad Sci USA. 88(21):9695-9 (1991)). In some aspects, alpha-L-iduronidase comprises a protein encoded by a nucleic acid comprising human alpha-L-iduronidase gene, for example, human alpha-L-iduronidase gene defined by GenBank Accession No. NM_000203 or NM_001363576. In some aspects, alpha-L-iduronidase comprises a protein comprising the amino acid sequence defined by GenBank Accession No. NP_000194 or NP_001350505.

The terms “iduronate 2-sulfatase,” “IDS,” “chondroitinsulfatase,” “iduronide-2-sulfate sulfatase,” “L-iduronosulfatase,” “L-idurono sulfate sulfatase,” “iduronate sulfatase,” “sulfo-L-iduronate sulfatase,” “L-iduronate 2-sulfate sulfatase,” “sulfoiduronate sulfohydrolase,” “2-sulfo-L-iduronate 2-sulfatase,” “iduronate-2-sulfate sulfatase,” “iduronate sulfate sulfatase,” and “L-iduronate-2-sulfate 2-sulfohydrolase” are used interchangeably and refer to a sulfatase enzyme which catalyses hydrolysis of the 2-sulfate groups of the L-iduronate 2-sulfate units of dermatan sulfate, heparan sulfate and heparin. In some aspects, iduronate 2-sulfatase comprises the enzyme described by IUBMB Enzyme Nomenclature EC 3.1.6.13 (as described, for example, in Archer et al., Biochim Biophys Acta 708:134-40 (1982)). In some aspects, iduronate 2-sulfatase comprises a protein encoded by a nucleic acid comprising human iduronate 2-sulfatase gene, for example, human iduronate 2-sulfatase gene defined by GenBank Accession No. NM_000202, NM_001166550, or NM_006123. In some aspects, iduronate 2-sulfatase comprises a protein comprising the amino acid sequence defined by GenBank Accession No. NP_000193, NP_001160022, or NP_006114.

The terms “sulfamidase,” “sulphamidase,” “N-sulfoglucosamine sulfohydrolase,” “SGSH,” “N-sulfo-D-glucosamine sulfohydrolase,” “sulfoglucosamine sulfamidase,” “heparin sulfamidase,” “2-desoxy-D-glucoside-2-sulphamate sulphohydrolase,” “sulphamate,” and sulphohydrolase” are used interchangeably and refer to an enzyme involved in the lysosomal degradation of heparan sulfate. This enzyme belongs to the family of hydrolases, specifically those acting on sulfur-nitrogen bonds. In some aspects, comprises the enzyme described by IUBMB Enzyme Nomenclature EC 3.10.1.1 (as described, for example, in Dietrich CP. Biochem J 111:91-5 (1969); Mahuran et al., Biochim Biophys Acta 757:359-65 (1983)). In some aspects, sulfamidase comprises a protein encoded by a nucleic acid comprising human sulfamidase gene, for example, human sulfamidase gene defined by GenBank Accession No. NM_000199, NM_001352921, or NM_001352922. In some aspects, sulfamidase comprises a protein comprising the amino acid sequence defined by GenBank Accession No. NP_000190, NP_001339850, or NP_001339851.

The terms “galactosamine-6-sulfatase,” “N-acetylgalactosamine-6-sulfatase”

“GALNS,” “galactosamine (N-acetyl)-6-sulfatase,” “chondroitin sulfatase,” “chondroitinase,” “galactose-6-sulfate sulfatase,” “acetylgalactosamine 6-sulfatase,” And “N-acetylgalactosamine-6-sulfate sulfatase” are used interchangeably and refer to a lysosomal exohydrolase required for the degradation of the glycosaminoglycans keratan sulfate and chondroitin 6-sulfate. In some aspects, galactosamine-6-sulfatase comprises the enzyme described by IUBMB Enzyme Nomenclature EC 3.1.6.4 (as described, for example, in et al., (1991)). In some aspects, galactosamine-6-sulfatase comprises a protein encoded by a nucleic acid comprising human galactosamine-6-sulfatase gene, for example, human galactosamine-6-sulfatase gene defined by GenBank Accession No. NM_000512, NM_001323543, or NM_001323544. In some aspects, galactosamine-6-sulfatase comprises a protein comprising the amino acid sequence defined by GenBank Accession No. NP_000503, NP_001310472, or NP_001310473.

The terms “N-acetylgalactosamine-4 sulfatase,” N-acetylgalactosamine-4-sulfatase,” “chondroitinsulfatase,” “chondroitinase,” “arylsulfatase B,” “acetylgalactosamine 4-sulfatase,” “N-acetylgalactosamine 4-sulfate sulfohydrolase,” and “N-acetyl-D-galactosamine-4-sulfate 4-sulfohydrolase” are used interchangeably and refer to an arylsulfatase that catalyzes the hydrolysis of the 4-sulfate groups of the N-acetyl-D-galactosamine 4-sulfate units of chondroitin sulfate and dermatan sulfate. In some aspects, e comprises the enzyme described by IUBMB Enzyme Nomenclature EC 3.1.6.12 (as described, for example, in Farooqui et al., Experientia 32:1242-1244 (1976)). In some aspects, N-acetylgalactosamine-4 sulfatase comprises a protein encoded by a nucleic acid comprising human N-acetylgalactosamine-4 sulfatase gene, for example, human N-acetylgalactosamine-4 sulfatase gene defined by GenBank Accession No. NM_00046. In some aspects, N-acetylgalactosamine-4 sulfatase comprises a protein comprising the amino acid sequence defined by GenBank Accession No. NC_000005 or NM_198709.

The terms “beta-glucuronidase,” “β-glucuronidase,” “β-glucuronide glucuronohydrolase glucuronidase,” “β-D-glucuronoside glucuronosohydrolase,” “exo-β-D-glucuronidase,” and “ketodaseare” used interchangeably and refer to an enzyme that catalyzes hydrolysis of β-D-glucuronic acid residues from the non-reducing end of mucopolysaccharides (also referred to as glycosaminoglycans) such as heparan sulfate. In some aspects, beta-glucuronidase comprises the enzyme described by IUBMB Enzyme Nomenclature EC 3.2.1.31 (as described, for example, in Diez et al., L. Eur. J. Biochem. 93:301-311 (1978)). In some aspects, beta-glucuronidase comprises a protein encoded by a nucleic acid comprising the beta-glucuronidase gene, for example, human beta-glucuronidase gene defined by GenBank Accession No. NM_000181, NM_001284290, NM_001293104, or NM_001293105. In some aspects, beta-glucuronidase comprises a protein comprising the amino acid sequence defined by GenBank Accession No. NP_000172, NP_001271219, or NP_001280034.

The terms “N-acetylglucosamine-1-phosphotransferase” and “N-acetylglucosamine-1-phosphate transferase” are used interchangeably and refer to an enzyme which catalyzes the first step in synthesis of a mannose 6-phosphate lysosomal recognition marker. In some aspects, N-acetylglucosamine-1-phosphotransferase comprises the enzyme described by IUBMB Enzyme Nomenclature EC 2.7.8.17 (as described, for example, in Nishikawa, A. Lysosomal Enzyme GlcNAc-1-Phosphotransferase. In Handbook of Glycosyltransferases and Related Genes. Springer, Tokyo (2002)). In some aspects, N-acetylglucosamine-1-phosphotransferase comprises a protein encoded by a nucleic acid comprising human N-acetylglucosamine-1-phosphotransferase gene, for example, human N-acetylglucosamine-1-phosphotransferase gene defined by GenBank Accession No. NM_032520, NM_024312, XM_011538731, or XM_006719593. In some aspects, N-acetylglucosamine-1-phosphotransferase comprises a protein comprising the amino acid sequence defined by GenBank Accession No. NP_115909, NP_077288, XP_011537033, or XP_006719656.

The terms “Niemann-Pick C1 protein,” “NPC intracellular cholesterol transporter 1,” and “NPC1” are used interchangeably and refer to an enzyme which resides in the limiting membrane of endosomes and lysosomes and mediates intracellular cholesterol trafficking via binding of cholesterol to its N-terminal domain. (Xiaochun et al., Proc Natl Acad Sci. 113(29):8212-7 (2016)). In some aspects, Niemann-Pick C1 protein comprises a protein encoded by a nucleic acid comprising human Niemann-Pick C1 protein gene, for example, human Niemann-Pick C1 protein gene defined by GenBank Accession No. NM_000271, XM_005258279, XM_005258277, XM_017025787, XM_006722479, XM_047437539, XM_017025786, XM_017025785, XM_017025784, or XM_005258278. In some aspects, Niemann-Pick C1 protein comprises a protein comprising the amino acid sequence defined by GenBank Accession No. NP_000262, XP_005258336, XP_005258334, XP_016881276, XP_006722542, XP_047293495, XP_016881275, XP 016881274, XP 016881273, or XP 005258335.

The terms “hexosaminidase A,” “beta-acetylaminodeoxyhexosidase,” “N-acetyl-beta-D-hexosaminidase,” “N-acetyl-beta-hexosaminidase,” “N-acetyl hexosaminidase,” “beta-hexosaminidase,” “beta-acetylhexosaminidinase,” “beta-D-N-acetylhexosaminidase,” “beta-N-acetyl-D-hexosaminidase,” “beta-N-acetylglucosaminidase,” “hexosaminidase A,” “N-acetylhexosaminidase,” “beta-D-hexosaminidase,” “β-N-acetylhexosaminidase,” and “HEXA” are used interchangeably and refer to an enzyme involved in the hydrolysis of terminal N-acetyl-D-hexosamine residues in N-acetyl-β-D-hexosaminides. In some aspects, hexosaminidase A comprises the enzyme described by IUBMB Enzyme Nomenclature EC 3.2.1.52 (as described, for example, in Tse et al., Biochemistry. 35(23):7599-607 (1996)). In some aspects, hexosaminidase A comprises a protein encoded by a nucleic acid comprising human hexosaminidase A gene, for example, human hexosaminidase A gene defined by GenBank Accession No. NM_000520 or NM_001318825. In some aspects, hexosaminidase A comprises a protein comprising the amino acid sequence defined by GenBank Accession No. NP_000511 or NP_001305754.

The terms, “tripeptidly peptidase 1,” “TPP1,” and “lysosomal pepstatin-insensitive proteaseare,” are used interchangeably and refer to a lysosomal serine protease. In some aspects, tripeptidly peptidase 1 comprises the enzyme described by IUBMB Enzyme Nomenclature EC 3.4.14.9 (as described, for example, in Ezaki et al., J. Neurochem. 72:2573-2582 (1999)). In some aspects, tripeptidly peptidase 1 comprises a protein encoded by a nucleic acid comprising human tripeptidly peptidase 1 gene, for example, human tripeptidly peptidase 1 gene defined by GenBank Accession No. NM_000391. In some aspects, tripeptidly peptidase 1 comprises a protein comprising the amino acid sequence defined by GenBank Accession No. NP_000382.

In some aspects, the lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) can be obtained from a cell endogenously expressing the lysosomal enzyme, or the lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) can be a recombinant human lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1), as described herein. In some aspects, the recombinant human lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) is a full length wild-type lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1). In some aspects, the recombinant human lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) comprises a subset of the amino acid residues present in a wild-type lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1), wherein the subset includes the amino acid residues of the wild-type lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) that form the active site for substrate binding and/or substrate reduction.

In some aspects, the recombinant human lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) is a fusion protein comprising the wild-type lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) active site for substrate binding and/or substrate reduction, as well as other amino acid residues that can or may not be present in the wild type recombinant human lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) recombinant human lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1).

The lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) can be obtained from commercial sources or can be obtained by synthesis techniques known to a person of ordinary skill in the art. The wild-type enzyme can be purified from a recombinant cellular expression system (e.g., mammalian cells such as CHO cells, or insect cells, see e.g., U.S. Pat. Nos. 5,580,757; 6,395,884; 6,458,574; 6,461,609; 6,210,666; 6,083,725), human placenta, or animal milk.

Other synthesis techniques for obtaining the lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) suitable for pharmaceutical use can be found, for example, in U.S. Pat. Nos. 7,560,424; 7,396,811; 423,135; 6,534,300; and 6,537,785; U.S. Published Application Nos. 2009/0203575; 2009/0029467; 2008/0299640; 2008/0241118; 2006/0121018; 2005/0244400; 2007/0280925; and 2004/0029779, and International Published Application No. 2005/077093.

In addition to proteins that comprise an amino acid sequence that is identical to the human lysosomal enzymes (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) described herein, this disclosure also encompasses the lysosomal enzymes (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) that are “substantially similar” thereto. Proteins described herein as being “substantially similar” to a reference protein include proteins that retain some structural and functional features of the native proteins yet differ from the native amino acid sequence at one or more amino acid positions (i.e., by amino acid substitutions).

Proteins altered from the native sequence can be prepared by substituting amino acid residues within a native protein and selecting proteins with the desired activity. For example, amino acid residues of the lysosomal enzyme, e.g., an α-Gal A protein can be systematically substituted with other residues and the substituted proteins can then be tested in standard assays for evaluating the effects of such substitutions on the ability of the protein to hydrolyze a terminal, non-reducing α-D-galactose residues in α-D-galactosides, including galactose oligosaccharides, galactomannans and galactolipids, and/or on the ability to treat or prevent Fabry disease.

In some aspects, to retain functional activity, conservative amino acid substitutions are made. As used herein, “conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In some aspects, a predicted nonessential amino acid residue in an α-Gal A protein is replaced with another amino acid residue from the same side chain family. Methods of identifying nucleotide and amino acid conservative substitutions which do not eliminate antigen binding are well-known in the art (see, e.g., Brummell et al., Biochem. 32: 1180-1187 (1993); Kobayashi et al. Protein Eng. 12(10):879-884 (1999); and Burks et al. Proc. Natl. Acad. Sci. USA 94:412-417 (1997)).

In some aspects, the lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) of the disclosure is at least about 70%, 71%, 72%, 73%, 74%, 75%, 76%, 77%, 78%, 79%, 80%, 81%, 82%, 83%, 84%, 85%, 86%, 87%, 88%, 89% 90%, 91%, 92%, 93%, 94%, 95%, 96%, 97%, 98%, or 99% identical to the amino acid sequence of the lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) described herein or known in the art.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules described herein. BLAST protein searches can be performed with the XBLAST program, score=50, word length=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See worldwideweb.ncbi.nlm.nih.gov.

An “antibody” (Ab) includes, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region is comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. The term “anti-lysosomal enzyme antibody,” for example, includes a full antibody having two heavy chains and two light chains that specifically binds to the lysosomal enzyme (α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) and antigen-binding portions of the full antibody.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to α-Gal A is substantially free of antibodies that bind specifically to antigens other than α-Gal A). An isolated antibody that binds specifically to α-Gal A can, however, have cross-reactivity to other antigens, such as α-Gal A molecules from different species. Moreover, an isolated antibody can be substantially free of other cellular material and/or chemicals.

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic or other techniques known to those skilled in the art.

The term “polyclonal antibodies” (pAbs) refers to a mixture of heterogeneous antibodies which are usually produced by different B cell clones in the body. They can recognize and bind to many different epitopes of a single antigen. In some aspects, the RP-01 antibody, described herein, is a polyclonal antibody.

A “human antibody” (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibody” and “fully human antibody” and are used synonymously.

A “humanized antibody” refers to an antibody in which some, most or all of the amino acids outside the CDRs of a non-human antibody are replaced with corresponding amino acids derived from human immunoglobulins. In one aspect of a humanized form of an antibody, some, most or all of the amino acids outside the CDRs have been replaced with amino acids from human immunoglobulins, whereas some, most or all amino acids within one or more CDRs are unchanged. Small additions, deletions, insertions, substitutions or modifications of amino acids are permissible as long as they do not abrogate the ability of the antibody to bind to a particular antigen. A “humanized antibody” retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen antibody” refers to an antibody that binds specifically to the antigen. For example, an anti-GAL antibody binds specifically to GAL; an anti-glucocerebrosidase antibody binds specifically to a glucocerebrosidase, an anti-alpha-glucosidase antibody binds specifically to an alpha-glucosidase, an anti-alpha-L-iduronidase antibody binds specifically to an alpha-L-iduronidase, an anti-iduronate 2-sulfatase antibody binds specifically to a iduronate 2-sulfatase, an anti-sulfamidase antibody binds specifically to a sulfamidase, an anti-galactosamine-6-sulfatase antibody binds specifically to a galactosamine-6-sulfatase, an anti-N-acetylgalactosamine-4 sulfatase antibody binds specifically to N-acetylgalactosamine-4 sulfatase, an anti-beta-glucuronidase antibody binds specifically to a beta-glucuronidase, an anti-N-acetylglucosamine-1-phosphotransferase antibody binds specifically to N-acetylglucosamine-1-phosphotransferase, an anti-Niemann-Pick C1 protein antibody binds specifically to a Niemann-Pick C1 protein, an anti-hexosaminidase A binds specifically to a hexosaminidase A, and an anti-tripeptidly peptidase 1 binds specifically to a tripeptidly peptidase 1.

The term “neutralizing anti-lysosomal enzyme antibody,” “anti-lysosomal enzyme NAb,” “neutralizing anti-drug antibody,” or “neutralizing ADA,” refers to an antibody that binds and inactivates (neutralizes) the lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1). For example, if the anti-lysosomal enzyme neutralizing antibodies (e.g., an anti-α-galactosidase A antibody, an anti-glucocerebrosidase antibody, an anti-alpha-glucosidase antibody, an anti-alpha-L-iduronidase antibody, an anti-iduronate 2-sulfatase antibody, an anti-sulfamidase antibody, an anti-galactosamine-6-sulfatase antibody, an anti-N-acetylgalactosamine-4 sulfatase antibody, an anti-beta-glucuronidase antibody, an anti-N-acetylglucosamine-1-phosphotransferase antibody, an anti-Niemann-Pick C1 protein antibody, an anti-hexosaminidase A, or an anti-tripeptidly peptidase 1 antibody) are present, the enzyme replacement therapy is directly inactivated (neutralized) by the anti-lysosomal enzyme neutralizing antibodies in the plasma, which can inhibit cellular uptake or enzymatic activity within the lysosome. In some aspects, if the anti-lysosomal enzyme neutralizing antibodies are present, they can neutralize the ERT activity by binding the enzyme (e.g., recombinant lysosomal enzyme).

In some aspects, the anti-lysosomal enzyme neutralizing antibody is an IgG antibody. In some aspects, the anti-lysosomal enzyme neutralizing antibody is an IgG4 antibody. In some aspects, the anti-lysosomal enzyme neutralizing antibody is an IgG2 antibody. In some aspects, the anti-lysosomal enzyme neutralizing antibody is an IgG1 antibody.

In some aspects, the anti-lysosomal enzyme neutralizing antibodies can develop within about one month, about two months, about three months, about four months, about five months, about six months, about seven months, about eight months, about nine months, about ten months, about eleven months, or within about twelve months of starting the enzyme replacement therapy.

The term “total anti-lysosomal enzyme antibody,” “anti-lysosomal enzyme TAb,” or “binding anti-lysosomal enzyme antibody” refers to the pre-existing anti-lysosomal enzyme antibodies (e.g., the anti-lysosomal enzyme antibodies present in the subject before the treatment (e.g., lysosomal storage disease treatment) (or before initiation of the clinical study)) and the treatment boosted antibodies (e.g., the pre-existing anti-lysosomal enzyme antibodies that were boosted to a higher level following administration of the lysosomal storage disease treatment). The terms “pre-existing anti-lysosomal enzyme antibodies” and “treatment boosted anti-lysosomal enzyme antibodies,” as used herein refer to the total anti-drug antibodies (ADAs) against the lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) in e.g., human serum, wherein the total ADA antibodies are detected, for example, using an enzyme-linked immunosorbent assay (ELISA) as descried in e.g., Partridge, M. A. et al., J Immunol Res.; 2016:6262383. (2016) or an electrochemiluminescence (ECL) type assay (e.g., Meso Scale Discovery (MSD)), as described in e.g., Partridge, M. A. et al., J Immunol Res.; 2016:6262383. (2016).

An “antigen-binding portion” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments of a full-length antibody. Examples of binding fragments encompassed within the term “antigen-binding portion” of an antibody, e.g., an anti-GLA antibody, an anti-glucocerebrosidase antibody, an anti-alpha-glucosidase antibody, an anti-alpha-L-iduronidase antibody, an anti-iduronate 2-sulfatase antibody, an anti-sulfamidase antibody, an anti-galactosamine-6-sulfatase antibody, an anti-N-acetylgalactosamine-4 sulfatase antibody, an anti-beta-glucuronidase antibody, an anti-N-acetylglucosamine-1-phosphotransferase antibody, an anti-Niemann-Pick C1 protein antibody, an anti-hexosaminidase A, or an anti-tripeptidly peptidase 1, described herein, include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ and is expressed as a molar concentration (M), whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} can be determined by techniques known to one of ordinary skill in the art, such as immunoassays (e.g., enzyme-linked immunosorbent assay (ELISA)), BIACORE®, BLI (Bio-layer interferometry), or kinetic exclusion assay (KINEXA®).

As used herein, the terms “specifically binds,” “specifically recognizes,” “specific binding,” “selective binding,” and “selectively binds,” are analogous terms in the context of antibodies and refer to molecules (e.g., antibodies) that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIACORE®, KINEXA® 3000 instrument (Sapidyne Instruments, Boise, ID), or other assays known in the art. In a specific aspect, molecules that specifically bind to an antigen bind to the antigen with a K_{A} that is at least 2 logs, 2.5 logs, 3 logs, 4 logs or greater than the K_{A} when the molecules bind to another antigen.

Antibodies typically bind specifically to their cognate antigen with high affinity, reflected by a dissociation constant (K_{D}) of 10⁻⁵ to 10⁻¹¹ M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, preferably 10⁻⁸ M or less, even more preferably 10⁻⁹ M or less, and most preferably between 10⁻⁸ M and 10⁻¹⁰ M or less, when determined by, e.g., immunoassays (e.g., ELISA) surface plasmon resonance (SPR) technology in a BIACORE™ 2000 instrument using the predetermined antigen, or BLI (Bio-layer interferometry) but does not bind with high affinity to unrelated antigens.

The term “nucleic acid molecule,” as used herein, is intended to include DNA molecules and RNA molecules. A nucleic acid molecule can be single- stranded or double-stranded, and can be cDNA.

The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

Nucleic acids, e.g., cDNA, can be mutated, in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Some vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, some vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses, adeno-associated viruses (“AAV”), and lentiviruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because some modifications can occur in succeeding generations due to either mutation or environmental influences, such progeny cannot, in fact, be identical to the parent cell, but are still included within the scope of the term “host cell” as used herein.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

The terms “lysosomal storage disease,” “lysosomal storage disorder,” “LSD,” “lysosomal storage diseases,” “lysosomal storage disorders,” and “LSDs” are used interchangeably and refer to a group of over 40 disorders which are the result of defects in genes encoding enzymes that break down glycolipid or polysaccharide waste products within the lysosomes of cells. The enzymatic products, e.g., sugars and lipids, are then recycled into new products. Each of these disorders results from an inherited autosomal or X-linked recessive trait which affects the levels of enzymes in the lysosome. Generally, there is no biological or functional activity of the affected enzymes in the cells and tissues of affected individuals. In some aspects, the lysosomal storage disease includes but is not limited to Fabry disease, Gaucher disease, Pompe disease, Mucopolysaccharidoses (MPS) type I disease, MPS type II, MPS type III, MPS type IV, MPS type VI, MPS type VII, mucolipidosis (ML), Niemann-Pick disease, Tay-Sachs disease, or Batten disease.

The term “Fabry disease” refers to classical Fabry disease, late-onset Fabry disease, and hemizygous females having mutations in the gene encoding an α-Gal A. The term “Fabry disease,” as used herein, further includes any condition in which a subject exhibits lower than normal endogenous α-Gal A activity. Fabry disease is referred to by many other names, for example, alpha-galactosidase A deficiency, Anderson-Fabry disease, angiokeratoma corporis diffusum, angiokeratoma diffuse, ceramide trihexosidase deficiency, Fabry's disease, GLA deficiency, and hereditary dystopic lipidosis. In some aspects, Fabry disease is type 1 classic phenotype or type 2 later-onset phenotype.

The term “Gaucher disease” refers to a rare, inherited metabolic disorder in which deficiency of the enzyme glucocerebrosidase results in the accumulation of harmful quantities of certain fats (lipids), specifically the glycolipid glucocerebroside, throughout the body especially within the bone marrow, spleen and liver. Gaucher disease is referred to by many other names, for example, cerebroside lipidosis syndrome, Gaucher splenomegaly, glucocerebrosidase deficiency, glucocerebrosidosis, glucosylceramidase deficiency, glucosyl cerebroside lipidosis, kerasin lipoidosis, kerasin thesaurismosis, lipid histiocytosis (kerasin type), and sphingolipidosis 1.

The term “Pompe disease” refers to a rare, multisystemic, hereditary disease in which deficiency of the enzyme alpha-glucosidase hampers the degradation of glycogen into glucose. Therefore, glycogen starts to accumulate in all kinds of tissues, but primarily in skeletal muscle, smooth muscle, and cardiac muscle, where it causes damage to tissue structure and function. Pompe disease is referred to by many other names, for example, glycogen storage disease type II (GSD II), acid maltase deficiency (AMD), and acid alpha-glucosidase (GAA) deficiency.

The term “mucopolysaccharidoses” or “MPS” refers a group of inherited lysosomal storage disorders.

“MPS type I disease” is characterized by a deficiency of the enzyme alpha-L-iduronidase, which results in an accumulation of dermatan and/or heparan sulfates. There are 3 types of MPS type I disease: Hurler syndrome (mucopolysaccharidosis type 1-H; MPS 1-H), Scheie syndrome (mucopolysaccharidosis type I-S; MPS 1-S), and Hurler-Scheie syndrome (mucopolysaccharidosis type I-H/S; MPS-IH/S).

“MPS type II disease” is a rare genetic disorder in which glycosaminoglycans (or GAGs or mucopolysaccharides) build up in body tissues. It is caused by a deficiency of the lysosomal enzyme iduronate-2-sulfatase (I2S). The lack of this enzyme causes heparan sulfate and dermatan sulfate to accumulate in all body tissues. It is the only type of MPS disorder inherited as an X-linked trait. MPS type II disease is referred to by other names, for example, Hunter syndrome, mucopolysaccharidosis type II, and MPS II.

“MPS type III disease” has four subtypes (A, B, C, and D) that are distinguished by four different enzyme deficiencies: heparan N-sulfatase, Alpha-N-acetylglucosaminidase, acetyl-CoA:alpha-glucosaminide N-acetyltransferase, and N-acetylglucosamine-6-sulfatase, respectively. MPS type III disease is referred to by other names, for example, Sanfilippo syndrome, mucopolysaccharidosis type III, and MPS III.

“MPS type IV disease” is a rare metabolic disorder in which the body cannot process glycosaminoglycans (AKA GAGs, or mucopolysaccharides). It is referred to by other names, for example, Morquio syndrome, mucopolysaccharidosis type Iv, and MPS IV. Morquio syndromes A and B occur because of a deficiency of the enzyme N-acetyl-galactosamine-6-sulfatase and beta-galactosidase, respectively, resulting in accumulation of keratan and chondroitin sulfate in type A and keratan sulfate in type B.

“MPS type VI disease” is characterized by a deficiency of the enzyme N-acetylgalactosamine-4-sulfatase, resulting in accumulation of dermatan sulfate. It is referred to by other names, for example, Maroteaux-Lamy syndrome, mucopolysaccharidosis type VI, and MPS VI.

“MPS type VII disease” is characterized by a deficiency of the enzyme beta-glucuronidase, resulting in the accumulation of three glycosaminoglycans: dermatan sulfate, heparan sulfate and chondroitin sulfate. It is referred to by other names, for example, Sly syndrome, mucopolysaccharidosis type VII, and MPS VII.

The term “mucolipidosis (ML)” refers to a group of inherited metabolic disorders that affect the body's ability to carry out the normal turnover of various materials within cells. Four conditions (types I, II, III, and IV) were historically labeled as mucolipidoses. However, type I (sialidosis) is now classified as a glycoproteinosis and type IV (Mucolipidosis type IV) is now classified as a gangliosidosis. Mucolipidosis types II and III (ML II and ML III) result from a deficiency of the enzyme N-acetylglucosamine-1-phosphotransferase, which phosphorylates target carbohydrate residues on N-linked glycoproteins. Without this phosphorylation, the glycoproteins are not destined for lysosomes, and they escape outside the cell.

“Niemann-Pick disease,” “Niemann Pick Disease Type C,” “NPC,” or “NPD-C” is a rare progressive genetic disorder characterized by an inability of the body to transport cholesterol and other fatty substances (lipids) inside of cells. It is caused in 95% of cases by loss of function of Niemann-Pick C1 protein (NPC1).

“Tay-Sachs disease” is a rare, neurodegenerative disorder in which deficiency of hexosaminidase A enzyme results in excessive accumulation of gangliosides in the brain and nerve cells. It is referred to by many other names, for example, HEXA deficiency, hexosaminidase A deficiency, TSD, GM2 gangliosidosis, type 1, hexoaminidase alpha-subunit deficiency (variant B), B variant GM2 gangliosidosis, sphingolipidosis, and Tay-Sachs.

“Batten disease” is the common name for a broad class of rare, fatal, inherited disorders of the nervous system also known as neuronal ceroid lipofuscinoses, or NCLs. It is a disorder in which the brain cells are missing an enzyme called tripeptidyl peptidase 1 (TPP1), which causes waste buildup in the cells' neurons. It is referred to by many other names, for example, Juvenile CLN3 Disease, CLN3, CLN3-NCL, JNCL, juvenile Batten disease, juvenile neuronal ceroid lipofuscinosis, neuronal ceroid lipofuscinosis 3, Spielmeyer-Sjogren disease, Vogt-Spielmeyer disease, and Vogt-Spielmeyer-Sjogren disease.

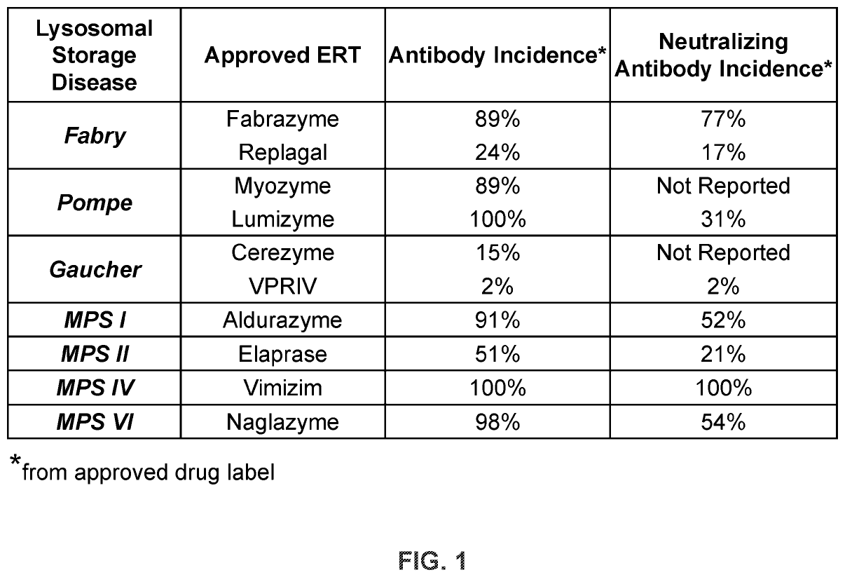

The term “enzyme replacement therapy” or “ERT” refers to the introduction of a non-native, purified enzyme into an individual having a deficiency in such enzyme (e.g., the lysosomal enzyme described herein). The administered enzyme can be obtained from natural sources or by recombinant expression. The term also refers to the introduction of a purified enzyme in an individual otherwise requiring or benefiting from administration of a purified enzyme, e.g., suffering from protein insufficiency. The introduced enzyme can be a purified, recombinant enzyme produced in vitro, or enzyme purified from isolated tissue or fluid, such as, e.g., placenta or animal milk, or from plants. FIG. 1 shows examples of currently approved enzyme replacement therapies (ERTs) for lysosomal storage disorders such as for example, Fabry disease, Pompe disease, Gaucher disease, MPS I, MPS II, MPS IV, and MPS VI.

The term “non-enzyme replacement therapy” refers to a therapy (e.g., the lysosomal storage disease therapy) that is not an enzyme replacement therapy. The non-enzyme replacement therapy can include small molecule therapy. Some emerging drug development strategies for small molecule therapy of the lysosomal storage disease include but are not limited to substrate reduction therapy (SRT), residual enzyme activation, protein homeostasis regulation (proteostasis), and pharmacological chaperone therapy (PCT).

The term “stabilize a proper conformation” refers to the ability of a compound or peptide or other molecule to associate with a wild-type protein, or to a mutant protein that can perform its wild-type function in vitro and in vivo, in such a way that the structure of the wild-type or mutant protein can be maintained as its native or proper form. This effect can manifest itself practically through one or more of (i) increased shelf-life of the protein; (ii) higher activity per unit/amount of protein; or (iii) greater in vivo efficacy. It can be observed experimentally through increased yield from the ER during expression; greater resistance to unfolding due to temperature increases (e.g., as determined in thermal stability assays), or the present of chaotropic agents, and by similar means.

As used herein, the term “active site” refers to the region of a protein that has some specific biological activity. For example, it can be a site that binds a substrate or other binding partner and contributes the amino acid residues that directly participate in the making and breaking of chemical bonds. Active sites in this application can encompass catalytic sites of enzymes, antigen biding sites of antibodies, ligand binding domains of receptors, binding domains of regulators, or receptor binding domains of secreted proteins. The active sites can also encompass transactivation, protein-protein interaction, or DNA binding domains of transcription factors and regulators.

As used herein, the term “active site-specific chaperone” refers to any molecule including a protein, peptide, nucleic acid, carbohydrate, etc. that specifically interacts reversibly with an active site of a protein and enhances formation of a stable molecular conformation. As used herein, “active site-specific chaperone” does not include endogenous general chaperones present in the ER of cells such as Bip, calnexin or calreticulin, or general, non-specific chemical chaperones such as deuterated water, DMSO, or TMAO.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms, “subject” and “patient” are used interchangeably herein.

The use of the term “flat dose” with regard to the methods and dosages of the disclosure means a dose that is administered to a patient without regard for the weight or body surface area (BSA) of the patient. The flat dose is therefore not provided as a mg/kg dose, but rather as an absolute amount of the agent (e.g., recombinant α-Gal A protein). For example, a 60 kg person and a 100 kg person would receive the same dose of an antibody (e.g., 12 mg of recombinant α-Gal A protein).

The term “weight-based dose” as referred to herein means that a dose that is administered to a patient is calculated based on the weight of the patient. For example, when a patient with 60 kg body weight requires 0.2 mg/kg of recombinant α-Gal A protein, one can calculate and use the appropriate amount of recombinant α-Gal A protein (i.e., 12 mg) for administration.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis).

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, an increase in overall survival (the length of time from either the date of diagnosis or the start of treatment for a disease, such as cancer, that patients diagnosed with the disease are still alive), or a prevention of impairment or disability due to the disease affliction. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

A “sample” or “biological sample” of the disclosure is of biological origin, in some aspects, such as from eukaryotic organisms. In some aspects, the sample is a human sample, but animal samples can also be used. Non-limiting sources of a sample for use in the present disclosure include solid tissue, biopsy aspirates, ascites, fluidic extracts, blood, plasma, serum, spinal fluid, lymph fluid, the external sections of the skin, respiratory, intestinal, and genitourinary tracts, tears, saliva, milk, tumors, organs, cell cultures and/or cell culture constituents, for example.

“Administering” refers to the physical introduction of a composition comprising a therapeutic agent to a subject, using any of the various methods and delivery systems known to those skilled in the art. Preferred routes of administration for recombinant α-Gal A protein or a gene expressing α-Gal A, include intravenous or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other non-parenteral routes include an oral, topical, epidermal or mucosal route of administration, for example, intranasally, vaginally, rectally, sublingually or topically. Administering can also be performed, for example, once, a plurality of times, and/or over one or more extended periods.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±three days, i.e., every eleven days to every seventeen days. Similar approximations apply, for example, to once about every three weeks, once about every four weeks, once about every five weeks, once about every six weeks, and once about every twelve weeks. In some aspects, a dosing interval of once about every six weeks or once about every twelve weeks means that the first dose can be administered any day in the first week, and then the next dose can be administered any day in the sixth or twelfth week, respectively. In other aspects, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Methods of the Disclosure

Provided herein are methods the methods of detecting an anti-lysosomal enzyme antibody in a human subject and the methods of improving an efficacy of an anti-lysosomal enzyme antibody assay, comprising pre-treating the biological sample of the subject with a base at pH of about 11 or greater and measuring the presence of the anti-lysosomal enzyme antibody in the biological sample of the subject.

In some aspects, the disclosure is directed to a method of detecting an anti-lysosomal enzyme antibody in a human subject (e.g., in the presence of a circulating lysosomal enzyme), comprising measuring the presence of the anti-lysosomal enzyme antibody in a biological sample of the subject, wherein the biological sample is pre-treated with a base at pH of about 11 or greater.

In some aspects, the disclosure is directed to a method of improving an efficacy of an anti-lysosomal enzyme antibody assay (e.g., improving the assay sensitivity and the circulating lysosomal enzyme tolerance), comprising pre-treating a biological sample of a human subject with a base at pH of about 11 or greater, further comprising measuring the presence of the anti-lysosomal enzyme antibody in the biological sample.

The term “improving an efficacy of an anti-lysosomal enzyme antibody assay” as used herein refers to any improvement resulting from optimizing various assay conditions (e.g., pre-treatment of the biological sample with a high or low molarity base at pH of about 11 or greater, heat pre-treatment of the biological sample, various on-board lysosomal enzyme concentrations (ng/ml), neutralizing the base pre-treated biological sample with an acid, or any combination thereof) that can result in an advantageous result (e.g., improving the assay sensitivity and the circulating lysosomal enzyme tolerance) as compared to the results achieved by the anti-lysosomal enzyme antibody assay when performed without the same assay conditions (e.g., pre-treatment of the biological sample with a high or low molarity base at pH of about 11 or greater, heat pre-treatment of the biological sample, various on-board lysosomal enzyme concentrations (ng/ml), neutralizing the base pre-treated biological sample with an acid, or any combination thereof). Enhanced effect and determination of enhanced effect can be measured by various parameters such as, but not limited to: improving the assay sensitivity measured by e.g., (i) increased ability to detect the anti-lysosomal enzyme neutralizing antibodies (NAbs) and/or total anti-lysosomal antibodies (TAbs) in the presence or absence of on-board lysosomal enzyme and/or (ii) improving the circulating lysosomal enzyme tolerance measured by e.g., increased tolerance of on-board lysosomal enzyme in detecting the anti-lysosomal enzyme NAbs as described in Examples 2-4 below.

### IIA. Anti-Lysosomal Enzyme Neutralizing Antibody (NAb) Assay

In some aspects, the disclosure is directed to methods of detecting an anti-lysosomal enzyme antibody (e.g., an anti-α-galactosidase A antibody, an anti-glucocerebrosidase antibody, an anti-alpha-glucosidase antibody, an anti-alpha-L-iduronidase antibody, an anti-iduronate 2-sulfatase antibody, an anti-sulfamidase antibody, an anti-galactosamine-6-sulfatase antibody, an anti-N-acetylgalactosamine-4 sulfatase antibody, an anti-beta-glucuronidase antibody, an anti-N-acetylglucosamine-1-phosphotransferase antibody, an anti-Niemann-Pick C1 protein antibody, an anti-hexosaminidase A, or an anti-tripeptidly peptidase 1 antibody) in a human subject and the methods of improving an efficacy of an anti-lysosomal enzyme antibody assay, comprising pre-treating the biological sample of the subject with a base at pH of about 11 or greater and measuring the presence of the anti-lysosomal enzyme antibody in the biological sample of the subject by the anti-lysosomal enzyme neutralizing antibody assay as described herein.

In some aspects, the anti-lysosomal antibody neutralizing antibody assay, as described in Example 2 below, determines the presence of the anti-lysosomal enzyme neutralizing antibodies (e.g., an anti-α-galactosidase A antibody, an anti-glucocerebrosidase antibody, an anti-alpha-glucosidase antibody, an anti-alpha-L-iduronidase antibody, an anti-iduronate 2-sulfatase antibody, an anti-sulfamidase antibody, an anti-galactosamine-6-sulfatase antibody, an anti-N-acetylgalactosamine-4 sulfatase antibody, an anti-beta-glucuronidase antibody, an anti-N-acetylglucosamine-1-phosphotransferase antibody, an anti-Niemann-Pick C1 protein antibody, an anti-hexosaminidase A, or an anti-tripeptidly peptidase 1 antibody) by assessing the neutralizing capacity of human serum on the lysosomal enzyme activity.

In some aspects, the results are determined by measuring a product resulting from cleavage of an artificial substrate, e.g., 4-Methylumbelliferyl-α-D-galactopyranoside, 4-Methylumbelliferyl-β-D-glucopyranoside, 4-Methylumbelliferyl α-D-glucopyranoside, 4-Methylumbelliferyl alpha-L-idopyranoside, 4-Methylumbelliferyl-α-L-Iduronide 2-sulfate, 4-Methylumbellieryl-2-sulfamino-2-deoxy-α-D-glucopyranoside,4-methylumbelliferyl-β-D-galactose-6-sulfate,4-Methylumbellieryl-N-acetyl-α-d-galactoseaminide-4-sulfate,4-methylumbelliferyl-3-d-glucuronide, 4-methylumbelliferyl-α-D-mannopyronoside, 4-Methylumbelliferyl N-acetyl-β-D-glucosaminide, or Ala-Ala-Phe-7-amido-4-methyl-coumarin.

In some aspects, the results are determined by measuring 4-methylumbelliferone (4-MU) product resulting from cleavage of an artificial substrate

Any anti-lysosomal enzyme neutralizing antibody present in the human serum will bind to the lysosomal enzyme and prevent the cleavage of the product from the substrate. This reduction in Relative Fluorescence Unit (RFU) signal is directly proportional to the amount of the anti-lysosomal enzyme neutralizing antibody (e.g., an anti-α-galactosidase A antibody, an anti-glucocerebrosidase antibody, an anti-alpha-glucosidase antibody, an anti-alpha-L-iduronidase antibody, an anti-iduronate 2-sulfatase antibody, an anti-sulfamidase antibody, an anti-galactosamine-6-sulfatase antibody, an anti-N-acetylgalactosamine-4 sulfatase antibody, an anti-beta-glucuronidase antibody, an anti-N-acetylglucosamine-1-phosphotransferase antibody, an anti-Niemann-Pick C1 protein antibody, an anti-hexosaminidase A, or an anti-tripeptidly peptidase 1 antibody) present in the human serum.

In some aspects, biological sample (e.g., a serum or plasma sample) is diluted at minimum required dilution (MRD) of about 2 fold or greater. In some aspects, the biological sample is diluted at MRD of about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 26 fold, about 27 fold, about 28 fold, about 29 fold, about 30 fold, about 31 fold, about 32 fold, about 33 fold, about 34 fold, about 35 fold, about 36 fold, about 37 fold, about 38 fold, about 39 fold, about 40 fold, about 41 fold, about 42 fold, about 43 fold, about 44 fold, about 45 fold, about 46 fold, about 47 fold, about 48 fold, about 49 fold, about 50 fold, about 51 fold, about 52 fold, about 53 fold, about 54 fold, about 55 fold, about 56 fold, about 57 fold, about 58 fold, about 59 fold, about 60 fold, about 61 fold, about 62 fold, about 63 fold, about 64 fold, about 65 fold, about 66 fold, about 67 fold, about 68 fold, about 69 fold, about 70 fold, about 71 fold, about 72 fold, about 73 fold, about 74 fold, about 75 fold, about 76 fold, about 77 fold, about 78 fold, about 79 fold, about 80 fold, about 81 fold, about 82 fold, about 83 fold, about 84 fold, about 85 fold, about 86 fold, about 87 fold, about 88 fold, about 89 fold, about 90 fold, about 91 fold, about 92 fold, about 93 fold, about 94 fold, about 95 fold, about 96 fold, about 97 fold, about 98 fold, about 99 fold, about 100 fold, about 200 fold, about 300 fold, about 400 fold, about 500 fold, about 600 fold, about 700 fold, about 800 fold, about 900 fold, about 1000 fold, about 1100 fold, about 1200 fold, about 1300 fold, about 1400 fold, about 1500 fold, about 1600 fold, about 1700 fold, about 1800 fold, about 1900 fold, about 2000 fold, about 2100 fold, about 2200 fold, about 2300 fold, about 2400 fold, about 2500 fold, about 2600 fold, about 2700 fold, about 2800 fold, about 2900 fold, about 3000 fold, about 3100 fold, about 3200 fold, about 3300 fold, about 3400 fold, about 3500 fold, about 3600 fold, about 3700 fold, about 3800 fold, about 3900 fold, about 4000 fold, about 4100 fold, about 4200 fold, about 4300 fold, about 4400 fold, about 4500 fold, about 4600 fold, about 4700 fold, about 4800 fold, about 4900 fold, or about 5000 fold.

In some aspects, the serum sample is diluted at MRD10 (1:5 in assay buffer (e.g., 0.1 M Citric Acid, 0.2 M Sodium Phosphate and 0.05% Triton X-100, pH 4.6+0.1) followed by 1:2 in 2× drug diluent (e.g., 40 ng/mL reconstituted GLA protein in an assay buffer)).

In some aspects, the lysosomal enzyme (e.g., α-galactosidase A (α-Gal A), glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) is at a concentration of at a concentration of less than about 10 ug/mL, less than about 9 ug/mL, less than about 8 ug/mL, less than about 7 ug/mL, less than about 6 ug/mL, less than about 5 ug/mL, less than about 4 ug/mL, less than about 3 ug/mL, less than about 2 ug/mL, less than about 1 ug/mL, less than about 0.9 ug/mL, less than about 0.8 ug/mL, less than about 0.7 ug/mL, less than about 0.6 ug/mL, less than about 0.5 ug/mL, less than about 0.4 ug/mL, less than about 0.3 ug/mL, less than about 0.2 ug/mL, less than about 100 ng/ml, less than about 90 ng/ml, less than about 80 ng/ml, less than about 70 ng/ml, less than about 60 ng/ml, less than about 50 ng/ml, less than about 40 ng/ml, less than about 30 ng/ml, less than about 20 ng/ml, or less than about 10 ng/ml.

In some aspects, the human lysosomal protein was diluted to 40 ng/mL in an acidic sample buffer containing 0.1M citric acid, 0.2M sodium phosphate, and 0.05% Triton X-100 at pH 4.6.

In some aspect the biological sample (e.g., a serum or plasma sample) is pre-treated with a base at pH of about 11 or greater.

In some aspects, the base is a non-buffered base. In some aspects, the non-buffered base is NaOH or Ca(OH)₂.

In some aspects, NaOH or Ca(OH)₂ is about 0.01M, about 0.02M, about 0.03M, about 0.04M, about 0.05M, about 0.06M, about 0.07M, about 0.08M, about 0.09M, or about 0.1M. In some aspects, NaOH or Ca(OH)₂ is about 0.02M.

In some aspects, pH is about 11, about 11.1, about 11.15, about 11.2, about 11.25, about 11.3, about 11.35, about 11.4, about 11.45, about 11.5, about 11.55, about 11.6, about 11.65, about 11.7, about 11.75, about 11.8, about 11.85, about 11.9, about 11.95, about 12, 12.1, about 12.15, about 12.2, about 12.25, about 12.3, about 12.35, about 12.4, about 12.45, about 12.5, about 12.55, about 12.6, about 12.65, about 12.7, about 12.75, about 12.8, about 12.85, about 12.9, about 12.95, about 13, 13.1, about 13.15, about 13.2, about 13.25, about 13.3, about 13.35, about 13.4, about 13.45, about 13.5, about 13.55, about 13.6, about 13.65, about 13.7, about 13.75, about 13.8, about 13.85, about 13.9, about 13.95, or about 14.

In some aspects, pH is greater than 11 and less than 12, greater than 11 and less than 13, or greater than 11 and less than 14.

In some aspects, human serum samples were diluted to 20% serum in a pH 12.45 alkaline sample buffer and then mixed 1:1 with the diluted acidic enzyme buffer. In some aspects, the final sample incubation concentration contained 20 ng/mL lysosomal enzyme at 10% serum, with a final pH of 4.9.

In some aspects, the pre-treated biological sample is mixed with a lysosomal enzyme.

In some aspects, the pre-treated biological sample and the lysosomal enzyme mixture is incubated for at least about one hour, at least about two hours, at least about three hours, at least about four hours, at least about five hours, at least about six hours, at least about seven hours, at least about eight hours, at least about nine hours, at least about ten hours, at least about eleven hours, at least about twelve hours, at least about thirteen hours, at least about fourteen hours, at least about fifteen hours, at least about sixteen hours, at least about seventeen hours, at least about eighteen hours, at least about nineteen hours, at least about twenty hours, at least about twenty one hours, at least about twenty two hours, at least about twenty three hours, or at least about twenty four hours.

In some aspects, the pre-treated biological sample and the lysosomal enzyme mixture is incubated for a duration between about 1 and about 15 hours, between about 2 and about 14 hours, between about 3 and about 13 hours, between about 4 and about 12 hours, between about 5 and about 11 hours, between about 6 and about 10 hours, or between about 7 and about 9 hours.

In some aspects, the pre-treated biological sample and the lysosomal enzyme are mixed with a reaction mix. In some aspects, the reaction mix comprises a substrate and/or an inhibitor.

In some aspects, the substrate is selected from the group consisting of: 4-Methylumbelliferyl-α-D-galactopyranoside, 4-Methylumbelliferyl-β-D-glucopyranoside, 4-Methylumbelliferyl α-D-glucopyranoside, 4-Methylumbelliferyl alpha-L-idopyranoside, 4-Methylumbelliferyl-α-L-Iduronide 2-sulfate, 4-Methylumbellieryl-2-sulfamino-2-deoxy-α-D-glucopyranoside,4-methylumbelliferyl-β-D-galactose-6-sulfate,4-Methylumbellieryl-N-acetyl-α-d-galactoseaminide-4-sulfate,4-methylumbelliferyl-β-d-glucuronide, 4-methylumbelliferyl-α-D-mannopyronoside, 4-Methylumbelliferyl N-acetyl-β-D-glucosaminide, and Ala-Ala-Phe-7-amido-4-methyl-coumarin.

In some aspects, the substrate is at a concentration of the substrate is at a concentration of at least about 1.1 mM, at least about 1.2 mM, at least about 1.3 mM, at least about 1.4 mM, at least about 1.5 mM, at least about 1.6 mM, at least about 1.7 mM, at least about 1.8 mM, at least about 1.9 mM, at least about 2 mM, at least about 2.1 mM, at least about 2.2 mM, at least about 2.3 mM, at least about 2.4 mM, at least about 2.5 mM, at least about 2.6 mM, at least about 2.7 mM, at least about 2.8 mM, at least about 2.9 mM, at least about 3 mM, at least about 3.1 mM, at least about 3.2 mM, at least about 3.3 mM, at least about 3.4 mM, at least about 3.5 mM, at least about 3.6 mM, at least about 3.7 mM, at least about 3.8 mM, at least about 3.9 mM, at least about 4 mM, at least about 4.1 mM, at least about 4.2 mM, at least about 4.3 mM, at least about 4.4 mM, at least about 4.5 mM, at least about 4.6 mM, at least about 4.7 mM, at least about 4.8 mM, at least about 4.9 mM, or at least about 5 mM. In some aspects, the substrate is at a concentration of at least about 2.5 mM.

In some aspects, the inhibitor comprises N-Acetylgalactosamine (GALNAc).

In some aspects, the inhibitor is at a concentration of less than about 200 mM, less than about 195 mM, less than about 190 mM, less than about 185 mM, less than about 180 mM, less than about 175 mM, less than about 170 mM, less than about 165 mM, less than about 160 mM, less than about 155 mM, less than about 150 mM, less than about 145 mM, less than about 140 mM, less than about 135 mM, less than about 130 mM, less than about 125 mM, less than about 120 mM, less than about 115 mM, or less than about 110 mM. In some aspects, the inhibitor is at a concentration of at least about 125 mM.

In some aspects, the reaction mix and the pre-treated biological sample with the lysosomal enzyme mixture are combined and mixed in a high throughput plate. In some aspects, the reaction mix and the pre-treated biological sample with the lysosomal enzyme mixture are incubated at room temperature at revolutions per minute (RPM) 400.

In some aspects, the method disclosed herein further comprises adding a stop buffer to the mixture after incubation. In some aspects, the incubation period is at least about 30 minutes, at least about 35 minutes, at least about 40 minutes, at least about 45 minutes, at least about 50 minutes, at least about 55 minutes, at least about 60 minutes, at least about 65 minutes, at least about 70 minutes, at least about 75 minutes, or at least about 80 minutes. In some aspects, the incubation period is at least about 60 minutes.

In some aspects, the stop buffer comprises glycine. In some aspects, the stop buffer is at a volume of less than about 1 mL, less than about 900 uL, less than about 800 uL, less than about 700 uL, less than about 600 uL, less than about 500 uL, less than about 400 uL, less than about 300 uL, less than about 200 uL, or less than about 100 uL. In some aspects, the stop buffer is at a volume of about 100 uL.

In some aspects, human biological samples having percent (%) inhibition equal to or greater than the cut-off point are identified as the anti-lysosomal enzyme neutralizing antibody positive, while those below the cut-off point are considered the anti-lysosomal enzyme neutralizing antibody negative.

In some aspects, the anti-lysosomal enzyme neutralizing antibody negative subject as described herein has a biological sample having lower than about 50% inhibition of the lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) activity as measured by an anti-lysosomal enzyme neutralizing antibody assay described herein. In some aspects, the anti-lysosomal enzyme neutralizing antibody negative sample as described herein has lower than about 1%, about 5%, about 10%, about 15%, about 20%, about 25% about 30%, about 35%, about 40%, about 45%, or about 50% inhibition of the lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) activity.

In some aspects, the anti-lysosomal enzyme neutralizing antibody positive subject as described herein has a biological sample having higher than about 10% inhibition of the lysosomal enzyme (e.g., α-Gal A, glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, or tripeptidly peptidase 1) activity as measured by an anti-lysosomal enzyme neutralizing antibody assay described herein. In some aspects, the anti-lysosomal enzyme neutralizing antibody positive sample as described herein has about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% signal inhibition.

### IIB. Standardization of an Anti-Lysosomal Enzyme Neutralizing Antibody (NAb) Assay

In some aspects, the disclosure is directed to a method of standardizing an anti-lysosomal enzyme neutralizing antibody assay comprising determining a lysosomal drug diluent concentration by measuring the effect of the lysosomal enzyme (e.g., α-Gal A) drug level on the inhibitory effect of the positive control antibody (e.g., an antibody designated as RP-01, 7H11, or 19D6), as described e.g., in Examples 2 and 3, below.

In some aspects, the positive control antibody includes, but is not limited to the RP-01 antibody. In some aspects, the RP-01 is a polyclonal antibody.

In some aspects, the positive control antibody includes, but is not limited to the 7H11 antibody. In some aspects, the 7H11 is a monoclonal antibody.

In some aspects, the positive control antibody includes, but is not limited to the 19D6 antibody. In some aspects, the 19D6 is a monoclonal antibody.

In some aspects, the standardization comprises determining a lysosomal enzyme drug diluent concentration by measuring the effect of the lysosomal enzyme (e.g., α-Gal A) drug level on the inhibitory effect of the RP-01 antibody (an anti-α-Gal antibody).

In some aspects, the standardization comprises determining a lysosomal enzyme drug diluent concentration by measuring the effect of the lysosomal enzyme (e.g., α-Gal A) drug level on the inhibitory effect of the 7H11 antibody (an anti-α-Gal antibody).

In some aspects, the standardization comprises determining a lysosomal enzyme drug diluent concentration by measuring the effect of the lysosomal enzyme (e.g., α-Gal A) drug level on the inhibitory effect of the 19D6 antibody (an anti-α-Gal antibody).

In some aspects, the lysosomal enzyme drug diluent concentration is less than about 500 ng/ml, less than about 400 ng/ml, less than about 300 ng/ml, less than about 200 ng/ml, less than about 150 ng/ml, less than about 145 ng/ml, less than about 140 ng/ml, less than about 135 ng/ml, less than about 130 ng/ml, less than about 125 ng/ml, less than about 120 ng/ml, less than about 110 ng/ml, less than about 105 ng/ml, less than about 100 ng/ml, less than about 90 ng/ml, less than about 80 ng/ml, less than about 70 ng/ml, less than about 60 ng/ml, less than about 50 ng/ml, less than about 40 ng/ml, less than about 30 ng/ml, less than about 20 ng/ml, or less than about 10 ng/ml. In some aspects, the lysosomal enzyme (e.g., α-Gal A) drug diluent concentration is less than about 125 ng/ml. In some aspects, the lysosomal enzyme (e.g., α-Gal A) drug diluent concentration is about 40 ng/ml. In some aspects, the lysosomal enzyme (e.g., α-Gal A) drug diluent concentration is about 20 ng/ml.

In some aspects, the inhibitory effect of the positive control antibody (RP-01, 7H11, or 19D6) is represented as about 10%, about 20%, about 30%, about 40%, about 50%, about 60%, about 70%, about 80%, or about 90% inhibition of α-Galactosidase A activity.

In some aspects, the GLA drug has a minimal required dilution (MRD) about 1 fold, about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 26 fold, about 27 fold, about 28 fold, about 29 fold, about 30 fold, about 31 fold, about 32 fold, about 33 fold, about 34 fold, about 35 fold, about 36 fold, about 37 fold, about 38 fold, about 39 fold, about 40 fold, about 41 fold, about 42 fold, about 43 fold, about 44 fold, about 45 fold, about 46 fold, about 47 fold, about 48 fold, about 49 fold, about 50 fold, about 51 fold, about 52 fold, about 53 fold, about 54 fold, about 55 fold, about 56 fold, about 57 fold, about 58 fold, about 59 fold, about 60 fold, about 61 fold, about 62 fold, about 63 fold, about 64 fold, about 65 fold, about 66 fold, about 67 fold, about 68 fold, about 69 fold, about 70 fold, about 71 fold, about 72 fold, about 73 fold, about 74 fold, about 75 fold, about 76 fold, about 77 fold, about 78 fold, about 79 fold, about 80 fold, about 81 fold, about 82 fold, about 83 fold, about 84 fold, about 85 fold, about 86 fold, about 87 fold, about 88 fold, about 89 fold, about 90 fold, about 91 fold, about 92 fold, about 93 fold, about 94 fold, about 95 fold, about 96 fold, about 97 fold, about 98 fold, about 99 fold, about 100 fold, about 200 fold, about 300 fold, about 400 fold, about 500 fold, about 600 fold, about 700 fold, about 800 fold, about 900 fold, about 1000 fold, about 1100 fold, about 1200 fold, about 1300 fold, about 1400 fold, about 1500 fold, about 1600 fold, about 1700 fold, about 1800 fold, about 1900 fold, about 2000 fold, about 2100 fold, about 2200 fold, about 2300 fold, about 2400 fold, about 2500 fold, about 2600 fold, about 2700 fold, about 2800 fold, about 2900 fold, about 3000 fold, about 3100 fold, about 3200 fold, about 3300 fold, about 3400 fold, about 3500 fold, about 3600 fold, about 3700 fold, about 3800 fold, about 3900 fold, about 4000 fold, about 4100 fold, about 4200 fold, about 4300 fold, about 4400 fold, about 4500 fold, about 4600 fold, about 4700 fold, about 4800 fold, about 4900 fold, or about 5000 fold.

In some aspects, the RP-01 antibody, the 7H11 antibody, or the 19D6 antibody has binding affinity (K_{D}) of less than 2.6×10⁻¹⁰ M, less than 2.5×10⁻¹⁰ M, less than 2.0×10⁻¹⁰ M, less than 1.5×10⁻¹⁰ M, less than 1.0×10⁻¹⁰ M, less than 9×10⁻¹¹ M, less than 8×10⁻¹¹ M, less than 7×10⁻¹¹ M, less than 6×10⁻¹¹ M, less than 5×10⁻¹¹ M, less than 4×10⁻¹¹ M, less than 3×10⁻¹¹ M, less than 2×10⁻¹¹ M, less than 1×10⁻¹¹ M, less than 9×10⁻¹² M, less than 8×10⁻¹² M, less than 7×10⁻¹² M, less than 6×10⁻¹² M, less than 5×10⁻¹² M, less than 4×10⁻¹² M, less than 3×10⁻¹² M, less than 2×10⁻¹² M, less than 1×10⁻¹² M, less than 9×10⁻¹³ M, or less than 8×10⁻¹³ M, when determined by, e.g., immunoassays (e.g., ELISA) surface plasmon resonance (SPR) technology in a BIACORE™ 2000 instrument using the predetermined antigen, or BLI (Bio-layer interferometry).

### IIC. Anti-Lysosomal Enzyme Total Antibody (TAb) Assay

In some aspects, the disclosure is directed to methods of detecting an anti-lysosomal enzyme antibody (e.g., an anti-α-galactosidase A antibody, an anti-glucocerebrosidase antibody, an anti-alpha-glucosidase antibody, an anti-alpha-L-iduronidase antibody, an anti-iduronate 2-sulfatase antibody, an anti-sulfamidase antibody, an anti-galactosamine-6-sulfatase antibody, an anti-N-acetylgalactosamine-4 sulfatase antibody, an anti-beta-glucuronidase antibody, an anti-N-acetylglucosamine-1-phosphotransferase antibody, an anti-Niemann-Pick C1 protein antibody, an anti-hexosaminidase A, or an anti-tripeptidly peptidase 1 antibody) in a human subject (e.g., in the presence of a circulating lysosomal enzyme) and the methods of improving an efficacy of an anti-lysosomal enzyme antibody assay (e.g., improving the assay sensitivity and the circulating lysosomal enzyme tolerance), comprising pre-treating the biological sample of the subject with a base at pH of about 11 or greater and measuring the presence of the anti-lysosomal enzyme antibody in the biological sample of the subject by the anti-lysosomal enzyme total antibody assay as described herein (e.g., Example 5 below).

In some aspects, the anti-lysosomal antibody total antibody assay, as described in Example 5 below, determines the presence of the anti-lysosomal enzyme neutralizing antibodies (e.g., an anti-α-galactosidase A antibody, an anti-glucocerebrosidase antibody, an anti-alpha-glucosidase antibody, an anti-alpha-L-iduronidase antibody, an anti-iduronate 2-sulfatase antibody, an anti-sulfamidase antibody, an anti-galactosamine-6-sulfatase antibody, an anti-N-acetylgalactosamine-4 sulfatase antibody, an anti-beta-glucuronidase antibody, an anti-N-acetylglucosamine-1-phosphotransferase antibody, an anti-Niemann-Pick C1 protein antibody, an anti-hexosaminidase A, or an anti-tripeptidly peptidase 1 antibody) by assessing the ability to detect serum anti-lysosomal antibodies in the presence of on-board lysosomal enzyme levels.

In some aspects, the methods disclosed herein further comprise neutralizing the pre-treated biological sample with an acid. In some aspects, the acid is acetic acid or hydrochloric acid. In some aspects, the acetic acid or hydrochloric acid is about 20 mM, about 30 mM, about 40 mM, about 50 mM, about 60 mM, about 70 mM, about 80 mM, about 90 mM, about 100 mM, about 200 mM, about 300 mM, about 400 mM, about 500 mM, or about 600 mM. In some aspects, the acetic acid is about 30 mM. In some aspects, the hydrochloric acid is about 120 mM.

In some aspects, a serum sample is pre-treated with a base at pH 11 for about 1 hour at room temperature and then neutralized with a 30 mM acetic acid.

In some aspects, the methods disclosed herein further comprise mixing the neutralized pre-treated biological sample with a lysosomal enzyme.

In some aspects, the neutralized pre-treated biological sample and the lysosomal enzyme mixture are added to a high throughput plate coated with a lysosomal enzyme antigen. In some aspects, the neutralized pre-treated biological sample and the lysosomal enzyme mixture are incubated by shaking for about 1 hour to about 2 hours at room temperature.

In some aspects, the lysosomal enzyme antigen is selected from a group consisting of: an α-Gal A antigen, a glucocerebrosidase antigen, an alpha-glucosidase antigen, an alpha-L-iduronidase antigen, a iduronate 2-sulfatase antigen, a sulfamidase antigen, a galactosamine-6-sulfatase antigen, a N-acetylgalactosamine-4 sulfatase antigen, a beta-glucuronidase antigen, a N-acetylglucosamine-1-phosphotransferase antigen, a Niemann-Pick C1 protein antigen, a hexosaminidase A antigen, and a tripeptidly peptidase 1 antigen. In some aspects, the lysosomal enzyme antigen is the α-Gal A antigen.

In some aspects, a detection antibody or lysosomal enzyme which has a ruthenylated label is added to the plate to produce a signal. In some aspects, the detection antibody is an anti-human IgG antibody. In some aspects, the ruthenylated anti-human IgG is added to the plate, incubated for an hour by shaking at room temperature.

In some aspects, the plate is read in a plate reader which detects light emissions by the ruthenylated label and outputs electrochemiluminescent units (ECLu). In some aspects, the ECLu represent the total levels of the anti-lysosomal total antibodies.

### III. Kits

Also within the scope of the present disclosure are kits comprising the anti-lysosomal enzyme neutralizing antibody assay, as described herein, wherein the kit comprises:
(a) an assay buffer; (b) a substrate; (c) GALNAc inhibitor; (d) stop solution; and (e) an insert comprising instructions for use of the kit.

Kits typically include a label indicating the intended use of the contents of the kit and instructions for use. The term label includes any writing, or recorded material supplied on or with the kit, or which otherwise accompanies the kit.

All of the references cited above, as well as all references cited herein, are incorporated herein by reference in their entireties.

The following examples are offered by way of illustration and not by way of limitation.

### EXAMPLES

### Example 1

### Anti-Lysosomal Enzyme Antibody Assays

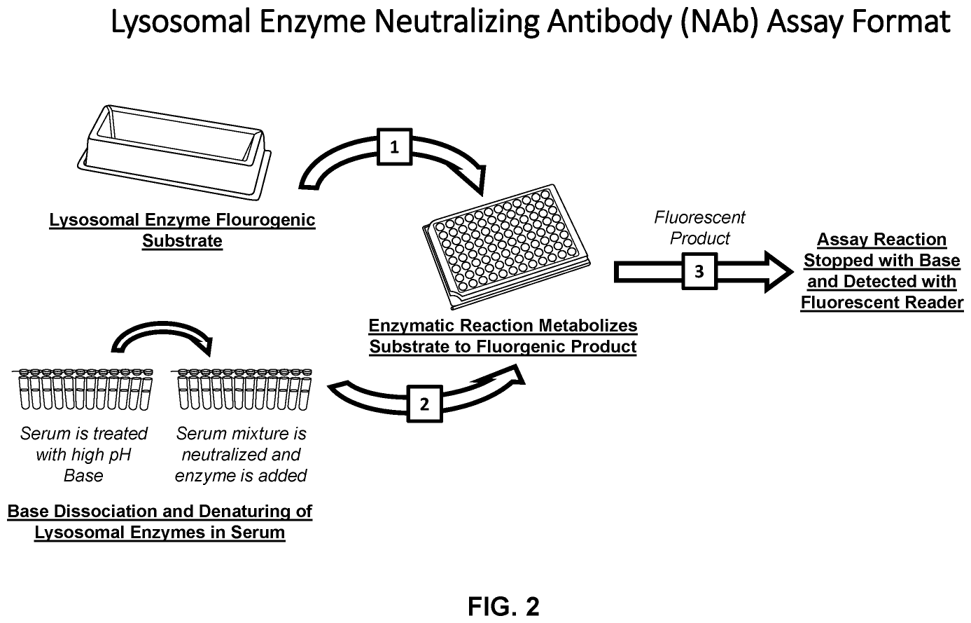

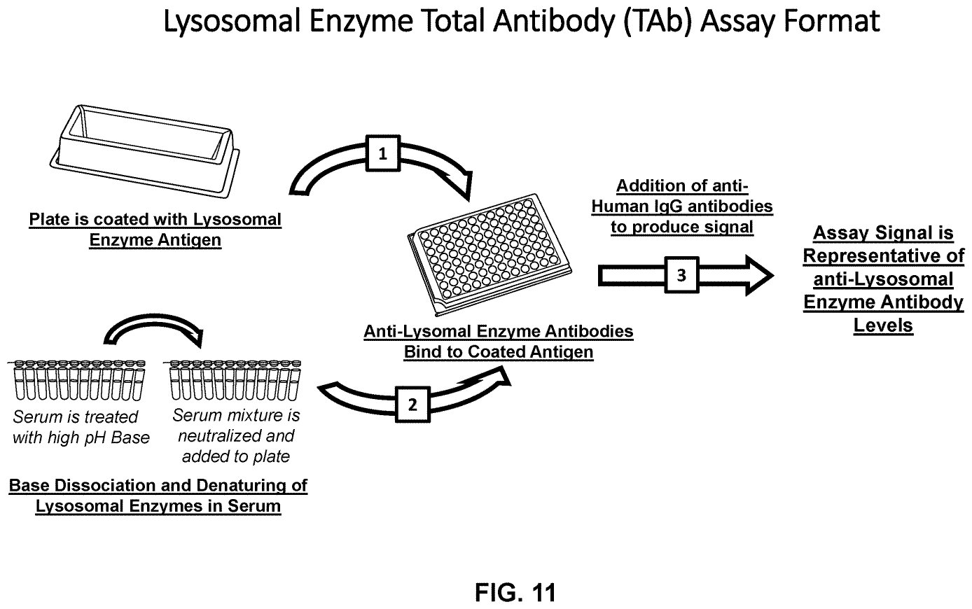

There is significant immunogenicity towards current treatments for lysosomal storage disorders (LSDs), and therefore it is important to be able to measure anti-lysosomal enzyme antibodies in patients to understand how and if new drugs will work in these patients. FIG. 1 shows that for lysosomal storage disorders (e.g., Fabry disease, Pompe disease, Gaucher disease, and Mucopolysaccharidoses (MPS) type I disease, MPS type II disease, MPS type IV disease, and MPS type VI disease) with currently approved enzyme replacement therapies (ERTs), there is a significant incidence of anti-ERT antibodies including anti-ERT neutralizing antibodies. Methods as shown in FIG. 2 and FIG. 11 allow for detection the anti-lysosomal neutralizing antibodies (FIG. 2) and the total anti-lysosomal antibodies (FIG. 11) in the presence of a circulating lysosomal enzyme. This is achieved through assay optimization, including a novel high pH alkaline treatment, which dissociates and specifically denatures lysosomal enzyme, allowing for accurate detection of the anti-lysosomal enzyme antibodies even in the presence of supraphysiological lysosomal enzyme levels in matrix such as serum and plasma. This alkaline assay method allows for the human subject's anti-lysosomal enzyme antibodies to be measured while, for example, being treated with ERT or gene therapy, allowing for the subject's stratification as well as understanding of the impact of antibody levels on safety and efficacy of treatments for lysosomal enzyme disorders including, for example, Fabry disease, Gaucher disease, Pompe disease, MPS I disease, MPS II disease, MPS VI disease, and Batten disease.

### Example 2

### Anti-Lysosomal Enzyme Neutralizing Antibody (NAb) Assay

The anti-lysosomal enzyme neutralizing antibody (NAb) assay utilized an artificial fluorogenic substrate, such as 4-methylumbelliferyl α-D-galactopyranoside (4-MU-α-Gal), to measure lysosomal enzyme activity and the inhibitory impact of neutralizing antibodies in human serum. Serum is treated with a high pH base treatment to dissociate lysosomal enzyme and anti-lysosomal enzyme antibody complexes, and further denature the lysosomal enzyme to eliminate catalytic enzymatic activity. The alkaline treated serum sample transferred to a constant low level of lysosomal enzyme, which is incubated with human serum overnight in an acidic assay buffer. After incubation, the sample was mixed with a fluorogenic substrate in a plate format. In the presence of catalytically active lysosomal enzyme, the substrate is cleaved and releases fluorescent 4-methylumbelliferone (4-MU) which can be quantitated at 365 nm excitation and 450 nm emission at basic pHs.

The presence of neutralizing antibodies in the serum limits substrate cleavage and diminishes relative fluorescence unit (RFU) signal. Each sample was normalized against the fluorescence observed in negative wells to determine the relative % inhibition of the sample

$$( \% Inhibition = [ 1 - ( \frac{Sample RFU}{Mean NC RFU} ) ] \times 100 ) .$$

A sample is considered to be anti-lysosomal neutralizing antibody positive if a sample inhibition (% inhibition of α-lysosomal enzyme activity) is greater than the calculated cut-off threshold.

The human lysosomal protein (α-galactosidase A) was diluted to 40 ng/mL in an acidic sample buffer (mimicking lysosomal-like conditions) containing 0.1M citric acid, 0.2M sodium phosphate, and 0.05% Triton X-100 at pH 4.6. Human serum samples were diluted to 20% serum in a pH 12.45 alkaline sample buffer (0.02M Ca(OH)2 (calcium hydroxide) buffer) and then mixed 1:1 with the diluted acidic enzyme buffer. The final sample incubation concentration contained 20 ng/mL lysosomal enzyme at 10% serum, with a final pH of 4.9. Positive controls were prepared by utilizing human sera with known concentration of anti-lysosomal enzyme neutralizing antibodies (e.g., RP-01, 7H11, or 19D6), while negative controls utilize human sera without the anti-lysosomal enzyme antibodies. The sample mixture is added in duplicate to a non-binding 96-well plate, sealed, and incubated overnight at 2-8° C.

After incubation, samples were transferred and diluted five-fold into a separate 96 well plate with reaction buffer containing specific lysosomal enzyme fluorogenic substrate and non-specific enzyme inhibitor such as 2.5 mM 4-MU-α-Gal and 0.125 M GALNAc respectively. These components are made in an acidic buffer containing 0.1M citric acid, 0.2M sodium phosphate and 0.05% Triton X-100 at pH 4.6. This reaction buffer continued to mimic lysosomal-like conditions while also containing specific lysosomal enzyme flourogenic substrate. The reaction occurred for one hour at room temperature. The reaction was terminated with addition of a basic glycine solution containing 0.25M glycine at pH 10.7. The plate was then analyzed in a spectrophotometer at 365 nm wavelength excitation and 450 nm wavelength emission. All samples were normalized and evaluated against negative control wells containing negative sera.

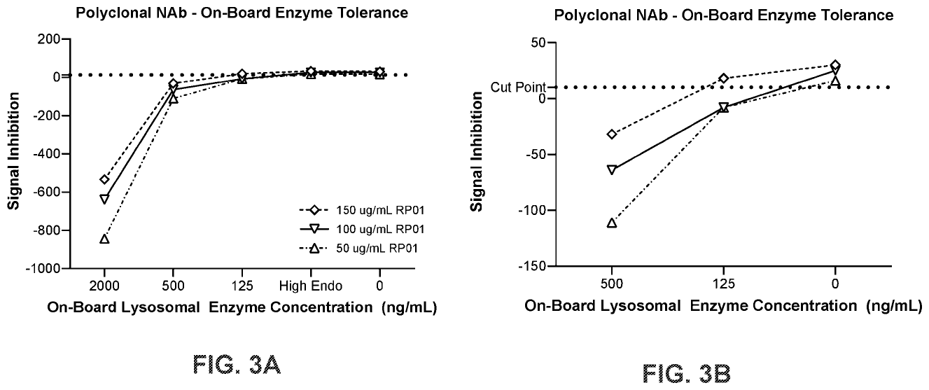

FIG. 3A and FIG. 3B show how the anti-lysosomal enzyme NAb assay is unable to detect antibodies accurately when 125 ng/mL of lysosomal enzyme (circulating α-galactosidase A) is present in serum. The assay shows complete loss of detection of any anti-lysosomal enzyme NAbs at lysosomal enzyme (α-galactosidase A) concentrations of 500 ng/ml or greater, with significant negative signal inhibition with increasing enzyme concentrations (FIG. 3A). FIG. 3A and FIG. 3B depict the inability to detect polyclonal lysosomal enzyme NAbs in the presence of on-board lysosomal enzyme in serum.

### Example 3

### Sample Pre-Treatments to Detect Anti-Lysosomal Enzyme Neutralizing Antibodies

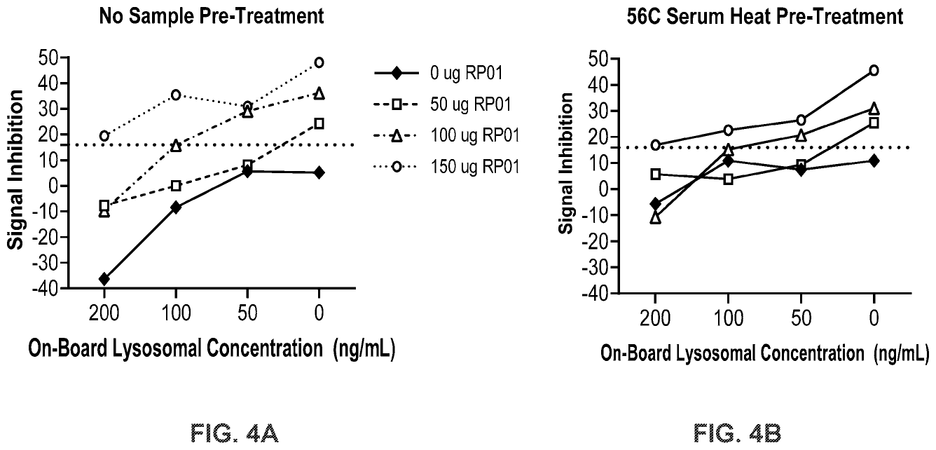

Different treatments were assessed to increase the ability to detect the anti-lysosomal enzyme neutralizing antibodies in the presence of on-board lysosomal enzyme. Heat pre-treatments were assessed by heating serum to attempt to dissociate and denature on-board lysosomal enzymes. As seen in FIG. 4A, the standard assay detects less NAb inhibition as on-board lysosomal enzyme (α-galactosidase A) increases to 200 ng/ml. In FIG. 4B, heat pre-treatment at 56° C. was able to denature on-board lysosomal enzyme (α-galactosidase A) when no NAb was present, but was unable to alleviate the interference at any NAb level (50 ug, 100 ug, or 150 ug).

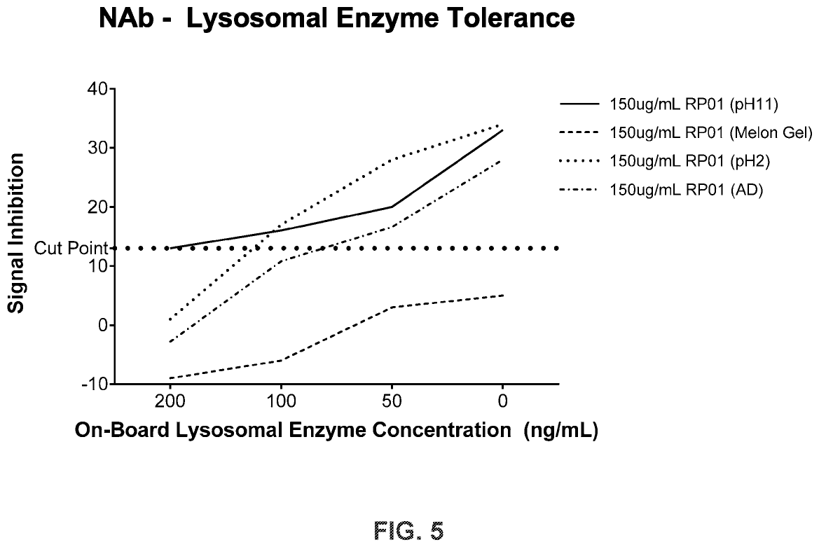

FIG. 5 shows other pre-treatment methods that were assessed to increase the ability to detect the anti-lysosomal enzyme NAbs in the presence of on-board lysosomal enzyme (α-galactosidase A). The acidic pH 2 pre-treatment was unable to increase tolerance of on-board lysosomal enzyme (α-galactosidase A) in detecting NAbs, and performed worse than the standard assay diluent (AD) condition (pH 4.5 McIlvaine buffer:0.1M Citric Acid+0.2M disodium hydrogen phosphate+0.05% TritonX). Melon Gel (Thermo Fisher Cat #45206) treatment was assessed as a method for purifying IgG from the serum sample and removing lysosomal enzyme. The Melon Gel approach was unsuccessful in increasing lysosomal enzyme tolerance, and lost the ability altogether to detect any NAbs even with no lysosomal enzyme on-board. The alkaline pH 11 treatment showed some increased tolerance to lysosomal enzyme in detecting NAbs, and performed the best of all assessed treatments in this experiment. The pH 11 pre-treatment showed improvement from the standard assay diluent but was not sufficient to detect all NAb inhibition when on-board lysosomal enzyme was increased.

### Example 4

### Optimized Alkaline Sample Pre-Treatment to Detect Anti-Lysosomal Enzyme Neutralizing Antibodies

The anti-lysosomal enzyme neutralizing antibody assay was optimized for lysosomal enzyme (α-galactosidase A) tolerance by testing the enzymatic neutralizing ability (% inhibition of lysosomal enzyme activity) of positive control antibodies (RP-01, 7H11, and 19D6) under various assay conditions.

The polyclonal RP-01 positive control antibody was generated as described in International Publ. No. WO 2022/072706. The monoclonal 7H11 and 19D6 rabbit monoclonal antibodies were generated by Yurogen Biosystems according to the following protocol. Rabbits were first immunized with the α-Gal A proteins. The spleen was isolated and resulting spleen cells were fused with partner cells to generate an immortal cell line that expresses antibodies (hybridoma technology). Hybridomas were screened for the most effective binding to the α-Gal A protein. The clones with the best binding affinity were expanded and purified. For screening to determine the best clones, plates are coated with alpha-Gal A, purified antibodies were diluted and plated onto the plate (lug/mL serially diluted 3× for 7 points), and HRP conjugated goat anti-rabbit IgG was used for final detection (1:5K).

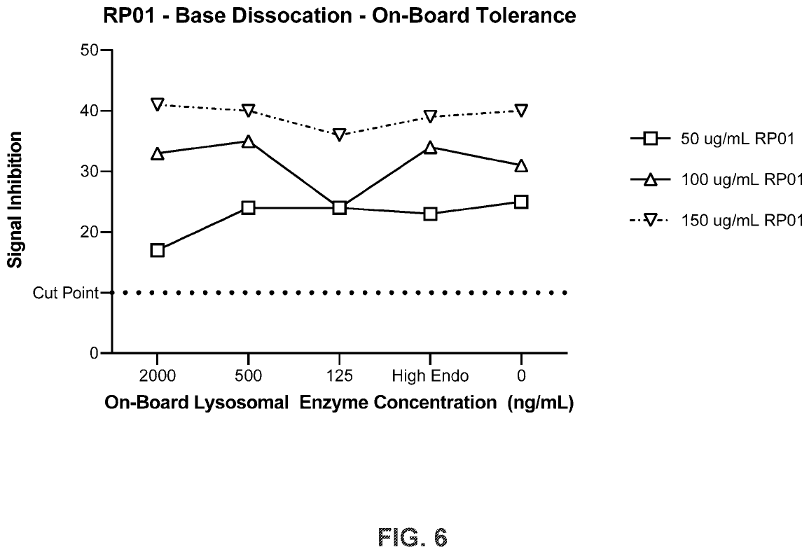

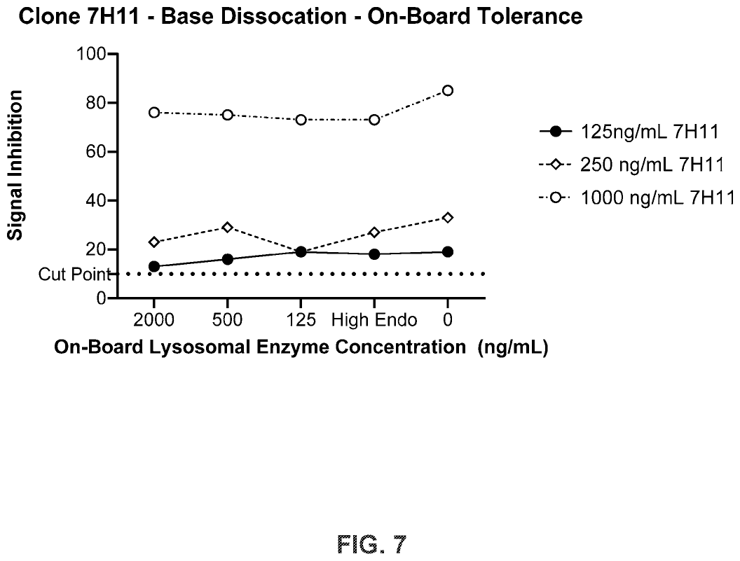

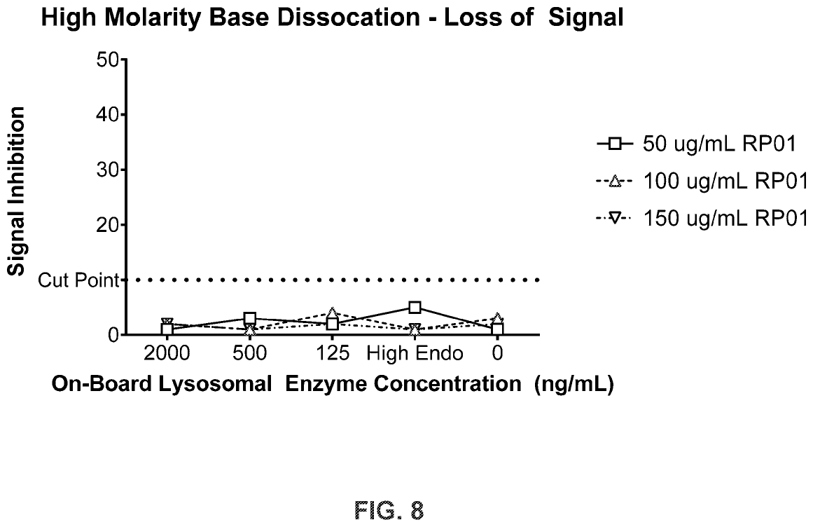

FIG. 6 shows that an alkaline sample pre-treatment was able to remove on-board lysosomal enzyme interference and dramatically increase the detect of the anti-lysosomal enzyme neutralizing antibodies. The NAb assay was able to tolerant up to 2000 ng/ml of on-board lysosomal enzyme (α-galactosidase A) at the 150 ug/mL, 100 ug/mL, and 50 ug/mL RP-01 polyclonal positive control levels. FIG. 7 shows that the alkaline sample pre-treatment increased on-board lysosomal enzyme (α-galactosidase A) tolerance up to 2000 ng/ml at 125 ng/mL, 250 ng/mL, and 1000 ng/mL 7H11 monoclonal positive control levels. The alkaline sample pre-treatment utilized was a low molarity pH 12.45 solution (0.02M Ca(OH)2 buffer) with limited buffering capacity. FIG. 8 shows that a higher molarity, buffered pH 12.45 solution (2M NaOH buffer) had negative effects on the assay and was unable to detect any NAb inhibition.

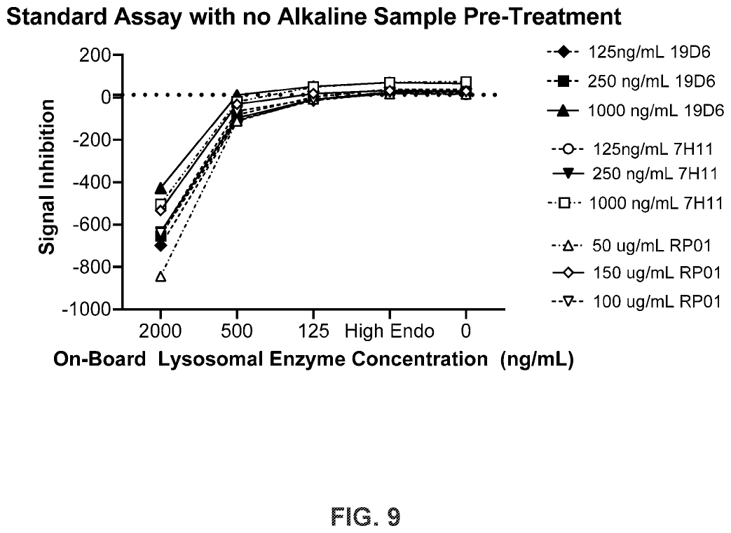

FIG. 9 shows that standard anti-lysosomal neutralizing antibody assay (which does not include the low molarity alkaline solution pre-treatment step) is not tolerant to high levels of serum lysosomal enzyme (α-galactosidase A) with any of the three positive controls assessed. Monoclonal antibodies 19D6 and 7H11 showed similar loss of NAb inhibition as polyclonal antibody RP-01 as on-board lysosomal enzyme increased above 125 ng/mL.

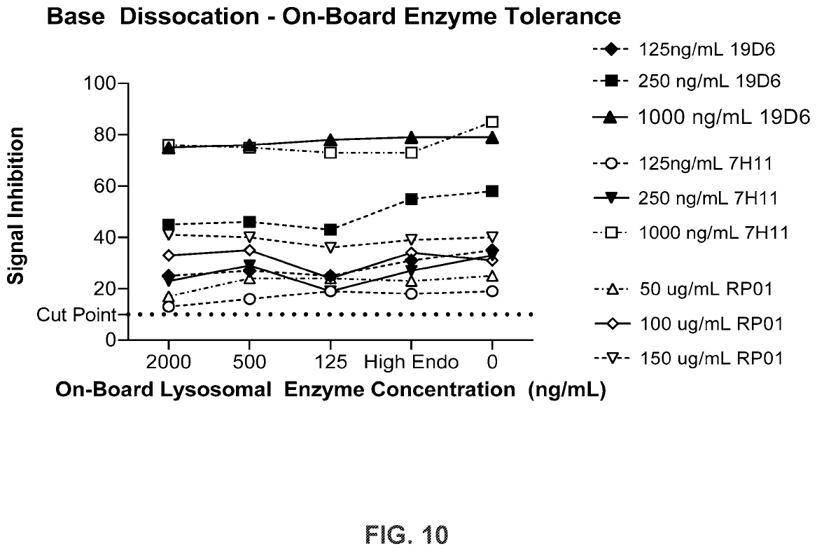

FIG. 10 shows that the anti-lysosomal neutralizing antibody assay, as described herein, is tolerant to high levels of serum lysosomal enzyme (α-galactosidase A) and is able to accurately measure NAb inhibition from three different positive controls (RP-01, 7H11, and 19D6).

### Example 5

### Anti-Lysosomal Enzyme Total Antibody (TAb) Assay

The anti-lysosomal enzyme total antibody assay was optimized for lysosomal enzyme tolerance by testing the binding capacity of 19D6 positive control antibody to plate-bound lysosomal enzyme (α-galactosidase A) in an enzyme-linked immunosorbent assay (ELISA) format as depicted in FIG. 11. A polystyrene ELISA plate is coated with 1 ug/mL of lysosomal enzyme in 1× phosphate-buffered saline (PBS) and incubated overnight at 2-8° C. Serum samples are pre-treated with a pH 11 alkaline solution, (10 uL sample+30 uL NaOH, 1 hr at room temperature) neutralized with an acidic solution (30 mM acetic acid), and further diluted with a neutral pH solution (assay buffer:1% BSA, 0.35 M NaCl, 0.25% CHAPS, 5 mM EDTA, 0.05% Tween x-100 or <5% BSA in PBS) to MRD40. These serum samples are then added to the overnight incubated ELISA plate to allow for the anti-lysosomal enzyme antibodies to bind to the lysosomal enzyme on the plate. After incubation and plate washing, a horse-radish peroxidase (HRP)-conjugated anti-human IgG detection antibody is then added to the plate which in the presence of TMB substrate is able to produce chromogenic signal relative to the amount of anti-lysosomal enzyme antibody present in the serum.

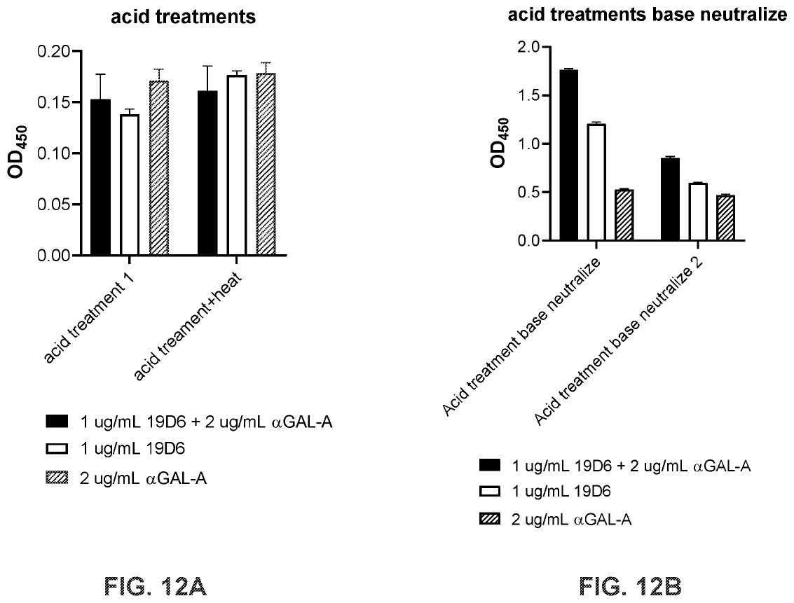

FIGS. 12A and 12B show various unsuccessful acidic pre-treatments that did not increase the ability to detect serum anti-lysosomal antibodies in the presence of on-board lysosomal enzyme (α-galactosidase A) levels. Acidic treatment with and without heat did not show any specific signal in the anti-lysosomal enzyme TAb assay (FIG. 12A). Acidic treatment with various alkaline neutralization was also unsuccessful in alleviating on-board lysosomal enzyme interference and exhibited high signal in serum samples without any specific anti-lysosomal enzyme antibody on-board (FIG. 12B).

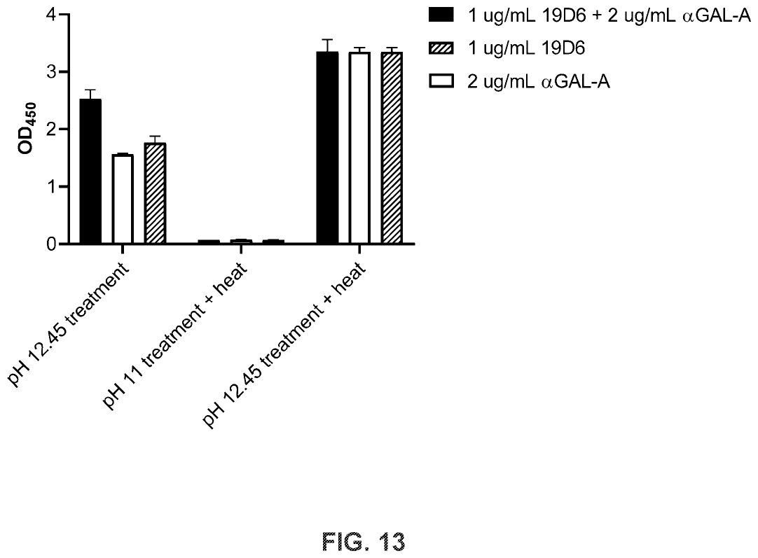

FIG. 13 shows various unsuccessful alkaline pre-treatments that did not increase the ability to detect serum anti-lysosomal antibodies in the presence of on-board lysosomal enzyme (α-galactosidase A) levels. Alkaline treatment at pH 12.45 with and without heat showed high non-specific signal in serum without any anti-lysosomal enzyme on-board. Alkaline treatment at pH 11 with heat treatment eliminated all signal in the assay and limited the ability detect any anti-lysosomal enzyme antibodies.

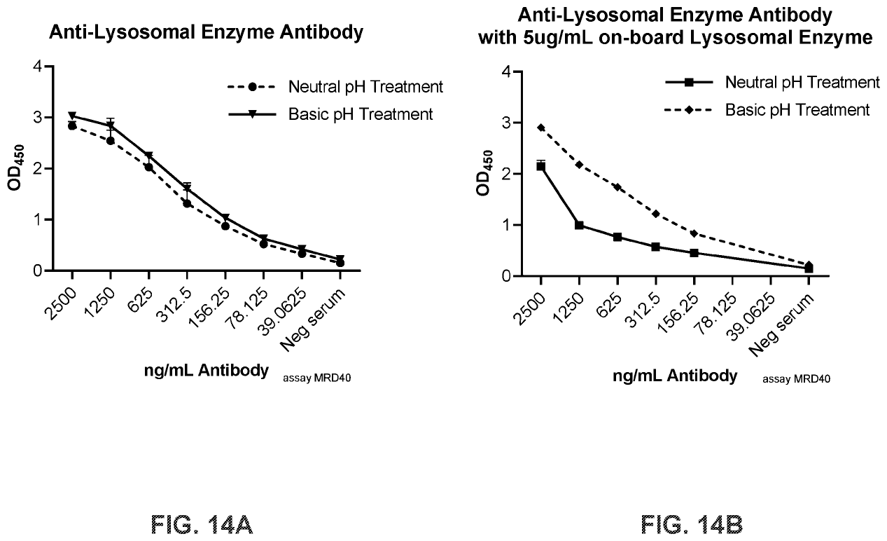

FIGS. 14A and 14B show that specific alkaline buffer sample pre-treatment (NaOH at pH 11) increases detection of the anti-lysosomal antibodies with 5 ug/mL of lysosomal enzyme on board (α-galactosidase A) from positive control antibody levels of 39.0625 ng/mL to 2500 ng/ml. Standard assay diluent at neutral pH shows significant loss in 450 nm optical density (OD) when lysosomal enzyme is on-board. This is dramatically improved after serum pre-treatment with pH 11 alkaline buffer, which recovers the ability to detect anti-lysosomal enzyme antibody levels in the presence of on-board serum lysosomal enzyme (5 ug/ml of α-galactosidase A) (FIG. 14B).

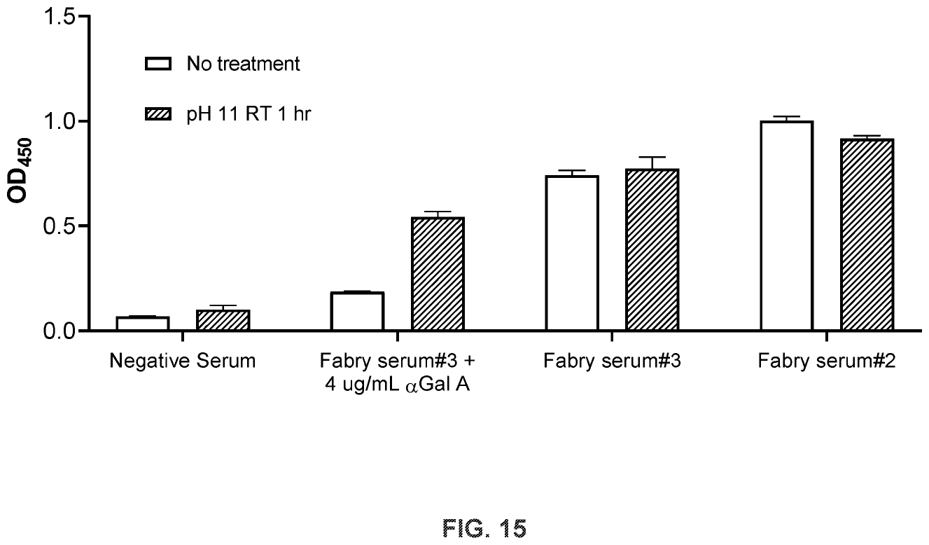

FIG. 15 shows that the pH 11 alkaline pre-treatment increases detection of anti-lysosomal enzyme antibodies in Fabry patient serum in the presence of 4ug/ml of on-board lysosomal enzyme (α-galactosidase A). The pH 11 alkaline treatment does not significantly change the sensitivity or detection of anti-lysosomal enzyme antibodies when there is no on-board lysosomal enzyme.

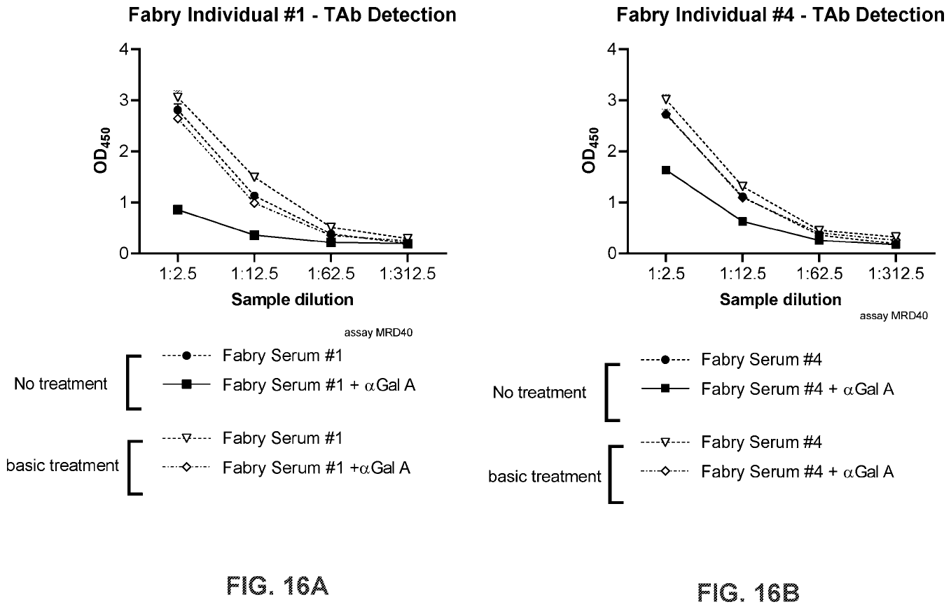

FIG. 16A and FIG. 16B show how alkaline base treatment (low molarity, (˜0.0001M) NaOH at pH 11) improves total antibody detection in Fabry patient serum, allowing for the assessment of antibody levels when on-board lysosomal enzyme (α-Galactosidase A) is present. For both Fabry individual #1 and Fabry individual #4, alkaline treatment increased optical density signal of samples with on-board lysosomal enzyme to levels similar to non-treated serum with no on-board lysosomal enzyme. This shows the successful use of the alkaline treatment to increase detection of anti-lysosomal enzyme antibodies in serum.

What is claimed is:

1. A method of detecting an anti-lysosomal enzyme antibody in a human subject, comprising measuring the presence of the anti-lysosomal enzyme antibody in a biological sample of the subject, wherein the biological sample is pre-treated with a base at pH of about 11 or greater.

2. A method of improving an efficacy of an anti-lysosomal enzyme antibody assay, comprising pre-treating a biological sample of a human subject with a base at pH of about 11 or greater, further comprising measuring the presence of the anti-lysosomal enzyme antibody in the biological sample.

3. The method of claim 1 or 2, wherein the base is a non-buffered base.

4. The method of claim 3, wherein the non-buffered base is NaOH or Ca(OH)₂.

5. The method of claim 4, wherein NaOH or Ca(OH)₂ is about 0.01M, about 0.02M, about 0.03M, about 0.04M, about 0.05M, about 0.06M, about 0.07M, about 0.08M, about 0.09M, or about 0.1M.

6. The method of claim 5, wherein NaOH or Ca(OH)₂ is about 0.02M.

7. The method of any one of claims 1 to 6, wherein the biological sample is a serum sample or a plasma sample.

8. The method of any one of claims 1 to 7, wherein the biological sample is diluted at minimum required dilution (MRD) of about 2 fold or greater.

9. The method of claim 8, wherein the biological sample is diluted at MRD of about 1 fold, 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 26 fold, about 27 fold, about 28 fold, about 29 fold, about 30 fold, about 31 fold, about 32 fold, about 33 fold, about 34 fold, about 35 fold, about 36 fold, about 37 fold, about 38 fold, about 39 fold, about 40 fold, about 41 fold, about 42 fold, about 43 fold, about 44 fold, about 45 fold, about 46 fold, about 47 fold, about 48 fold, about 49 fold, about 50 fold, about 51 fold, about 52 fold, about 53 fold, about 54 fold, about 55 fold, about 56 fold, about 57 fold, about 58 fold, about 59 fold, about 60 fold, about 61 fold, about 62 fold, about 63 fold, about 64 fold, about 65 fold, about 66 fold, about 67 fold, about 68 fold, about 69 fold, about 70 fold, about 71 fold, about 72 fold, about 73 fold, about 74 fold, about 75 fold, about 76 fold, about 77 fold, about 78 fold, about 79 fold, about 80 fold, about 81 fold, about 82 fold, about 83 fold, about 84 fold, about 85 fold, about 86 fold, about 87 fold, about 88 fold, about 89 fold, about 90 fold, about 91 fold, about 92 fold, about 93 fold, about 94 fold, about 95 fold, about 96 fold, about 97 fold, about 98 fold, about 99 fold, about 100 fold, about 200 fold, about 300 fold, about 400 fold, about 500 fold, about 600 fold, about 700 fold, about 800 fold, about 900 fold, about 1000 fold, about 1100 fold, about 1200 fold, about 1300 fold, about 1400 fold, about 1500 fold, about 1600 fold, about 1700 fold, about 1800 fold, about 1900 fold, about 2000 fold, about 2100 fold, about 2200 fold, about 2300 fold, about 2400 fold, about 2500 fold, about 2600 fold, about 2700 fold, about 2800 fold, about 2900 fold, about 3000 fold, about 3100 fold, about 3200 fold, about 3300 fold, about 3400 fold, about 3500 fold, about 3600 fold, about 3700 fold, about 3800 fold, about 3900 fold, about 4000 fold, about 4100 fold, about 4200 fold, about 4300 fold, about 4400 fold, about 4500 fold, about 4600 fold, about 4700 fold, about 4800 fold, about 4900 fold, or about 5000 fold.

10. The method of any one of claims 1 to 9, wherein pH is about 11, about 11.1, about 11.15, about 11.2, about 11.25, about 11.3, about 11.35, about 11.4, about 11.45, about 11.5, about 11.55, about 11.6, about 11.65, about 11.7, about 11.75, about 11.8, about 11.85, about 11.9, about 11.95, about 12, 12.1, about 12.15, about 12.2, about 12.25, about 12.3, about 12.35, about 12.4, about 12.45, about 12.5, about 12.55, about 12.6, about 12.65, about 12.7, about 12.75, about 12.8, about 12.85, about 12.9, about 12.95, about 13, 13.1, about 13.15, about 13.2, about 13.25, about 13.3, about 13.35, about 13.4, about 13.45, about 13.5, about 13.55, about 13.6, about 13.65, about 13.7, about 13.75, about 13.8, about 13.85, about 13.9, about 13.95, or about 14.

11. The method of claim 10, wherein pH is 12.45.

12. The method of any one of claims 1 to 9, wherein pH is greater than 11 and less than 12, greater than 11 and less than 13, or greater than 11 and less than 14.

13. The method of any one of claims 1 to 12, wherein the subject has a lysosomal storage disease.

14. The method of claim 13, wherein the lysosomal storage disease is selected from a group consisting of: Fabry disease, Gaucher disease, Pompe disease, Mucopolysaccharidoses (MPS) type I disease, MPS type II, MPS type III, MPS type IV, MPS type VI, MPS type VII, mucolipidosis (ML), Niemann-Pick disease, Tay-Sachs disease, and Batten disease.

15. The method of any one of claims 1 to 14, wherein the presence of an anti-lysosomal enzyme neutralizing antibody is measured.

16. The method of any one of claims 1 to 14, wherein the presence of anti-lysosomal enzyme total antibodies is measured.

17. The method of claim 15, further comprising mixing the pre-treated biological sample with a lysosomal enzyme.

18. The method of claim 17, wherein the lysosomal enzyme is selected from a group consisting of: α-galactosidase A (α-Gal A), glucocerebrosidase, alpha-glucosidase, alpha-L-iduronidase, iduronate 2-sulfatase, sulfamidase, galactosamine-6-sulfatase, N-acetylgalactosamine-4 sulfatase, beta-glucuronidase, N-acetylglucosamine-1-phosphotransferase, Niemann-Pick C1 protein, hexosaminidase A, and tripeptidly peptidase 1.

19. The method of claim 18, wherein the lysosomal enzyme is α-galactosidase A.

20. The method of any one of claims 17 to 19, wherein the neutralizing anti-lysosomal enzyme antibody is selected from a group consisting of: an anti-α-galactosidase A antibody, an anti-glucocerebrosidase antibody, an anti-alpha-glucosidase antibody, an anti-alpha-L-iduronidase antibody, an anti-iduronate 2-sulfatase antibody, an anti-sulfamidase antibody, an anti-galactosamine-6-sulfatase antibody, an anti-N-acetylgalactosamine-4 sulfatase antibody, an anti-beta-glucuronidase antibody, an anti-N-acetylglucosamine-1-phosphotransferase antibody, an anti-Niemann-Pick C1 protein antibody, an anti-hexosaminidase A, and an anti-tripeptidly peptidase 1 antibody.

21. The method of claim 20, wherein the neutralizing anti-lysosomal enzyme antibody is the anti-α-galactosidase A antibody.

22. The method of any one of claims 17 to 21, wherein the pre-treated biological sample and the lysosomal enzyme mixture is incubated for at least about one hour, at least about two hours, at least about three hours, at least about four hours, at least about five hours, at least about six hours, at least about seven hours, at least about eight hours, at least about nine hours, at least about ten hours, at least about eleven hours, at least about twelve hours, at least about thirteen hours, at least about fourteen hours, at least about fifteen hours, at least about sixteen hours, at least about seventeen hours, at least about eighteen hours, at least about nineteen hours, at least about twenty hours, at least about twenty one hours, at least about twenty two hours, at least about twenty three hours, or at least about twenty four hours.

23. The method of any one of claims 17 to 22, further comprising mixing the pre-treated biological sample and the lysosomal enzyme with a reaction mix.

24. The method of any one of claims 16 to 23, wherein the lysosomal enzyme is at a concentration of less than about 10 ug/mL, less than about 9 ug/mL, less than about 8 ug/mL, less than about 7 ug/mL, less than about 6 ug/mL, less than about 5 ug/mL, less than about 4 ug/mL, less than about 3 ug/mL, less than about 2 ug/mL, less than about 1 ug/mL, less than about 0.9 ug/mL, less than about 0.8 ug/mL, less than about 0.7 ug/mL, less than about 0.6 ug/mL, less than about 0.5 ug/mL, less than about 0.4 ug/mL, less than about 0.3 ug/mL, less than about 0.2 ug/mL, less than about 100 ng/ml, less than about 90 ng/ml, less than about 80 ng/ml, less than about 70 ng/ml, less than about 60 ng/ml, less than about 50 ng/ml, less than about 40 ng/ml, less than about 30 ng/ml, less than about 20 ng/ml, or less than about 10 ng/ml.

25. The method of any one of claims 17 to 24, wherein the pre-treated biological sample and the lysosomal enzyme mixture is incubated for at least about one hour, at least about two hours, at least about three hours, at least about four hours, at least about five hours, at least about six hours, at least about seven hours, at least about eight hours, at least about nine hours, at least about ten hours, at least about eleven hours, at least about twelve hours, at least about thirteen hours, at least about fourteen hours, at least about fifteen hours, at least about sixteen hours, at least about seventeen hours, at least about eighteen hours, at least about nineteen hours, at least about twenty hours, at least about twenty one hours, at least about twenty two hours, at least about twenty three hours, or at least about twenty four hours.

26. The method of any one of claims 17 to 24, wherein the pre-treated biological sample and the lysosomal enzyme mixture is incubated for a duration between about 1 and about 15 hours, between about 2 and about 14 hours, between about 3 and about 13 hours, between about 4 and about 12 hours, between about 5 and about 11 hours, between about 6 and about 10 hours, or between about 7 and about 9 hours.

27. The method of claim 23, wherein the reaction mix comprises a substrate and/or an inhibitor.

28. The method of claim 27, wherein the substrate is selected from the group consisting of: 4-Methylumbelliferyl-α-D-galactopyranoside, 4-Methylumbelliferyl-β-D-glucopyranoside, 4-Methylumbelliferyl α-D-glucopyranoside, 4-Methylumbelliferyl alpha-L-idopyranoside, 4-Methylumbelliferyl-α-L-Iduronide 2-sulfate, 4-Methylumbellieryl-2-sulfamino-2-deoxy-α-D-glucopyranoside,4-methylumbelliferyl-β-D-galactose-6-sulfate,4-Methylumbellieryl-N-acetyl-α-d-galactoseaminide-4-sulfate,4-methylumbelliferyl-3-d-glucuronide, 4-methylumbelliferyl-α-D-mannopyronoside, 4-Methylumbelliferyl N-acetyl-β-D-glucosaminide, and Ala-Ala-Phe-7-amido-4-methyl-coumarin.

29. The method of claim 27 or 28, wherein the substrate is at a concentration of at least about 1.1 mM, at least about 1.2 mM, at least about 1.3 mM, at least about 1.4 mM, at least about 1.5 mM, at least about 1.6 mM, at least about 1.7 mM, at least about 1.8 mM, at least about 1.9 mM, at least about 2 mM, at least about 2.1 mM, at least about 2.2 mM, at least about 2.3 mM, at least about 2.4 mM, at least about 2.5 mM, at least about 2.6 mM, at least about 2.7 mM, at least about 2.8 mM, at least about 2.9 mM, at least about 3 mM, at least about 3.1 mM, at least about 3.2 mM, at least about 3.3 mM, at least about 3.4 mM, at least about 3.5 mM, at least about 3.6 mM, at least about 3.7 mM, at least about 3.8 mM, at least about 3.9 mM, at least about 4 mM, at least about 4.1 mM, at least about 4.2 mM, at least about 4.3 mM, at least about 4.4 mM, at least about 4.5 mM, at least about 4.6 mM, at least about 4.7 mM, at least about 4.8 mM, at least about 4.9 mM, or at least about 5 mM.

30. The method of claim 27, wherein the inhibitor comprises N-Acetylgalactosamine (GALNAc).

31. The method of claim 27, where the inhibitor is at a concentration of less than about 200 mM, less than about 195 mM, less than about 190 mM, less than about 185 mM, less than about 180 mM, less than about 175 mM, less than about 170 mM, less than about 165 mM, less than about 160 mM, less than about 155 mM, less than about 150 mM, less than about 145 mM, less than about 140 mM, less than about 135 mM, less than about 130 mM, less than about 125 mM, less than about 120 mM, less than about 115 mM, or less than about 110 mM.

32. The method of any one of claims 23 to 31, wherein the reaction mix and the pre-treated biological sample with the lysosomal enzyme mixture are combined in a high throughput plate.

33. The method of claim 32, wherein the reaction mix and the pre-treated biological sample with the lysosomal enzyme mixture are incubated at room temperature at revolutions per minute (RPM) 300, 400, 500, or 600.

34. The method of claim 33, further comprising adding a stop buffer to the mixture after incubation.

35. The method of claim 33 or 34, wherein the incubation period is at least about 30 minutes, at least about 35 minutes, at least about 40 minutes, at least about 45 minutes, at least about 50 minutes, at least about 55 minutes, at least about 60 minutes, at least about 65 minutes, at least about 70 minutes, at least about 75 minutes, or at least about 80 minutes.

36. The method of claim 34 or 35, wherein the stop buffer comprises glycine.

37. The method of any one of claims 34 to 36, wherein the stop buffer is at a volume of less than about 1 mL, less than about 900 uL, less than about 800 uL, less than about 700 uL, less than about 600 uL, less than about 500 uL, less than about 400 uL, less than about 300 uL, less than about 200 uL, or less than about 100 uL.

38. The method of claim 16, further comprising neutralizing the pre-treated biological sample with an acid.

39. The method of claim 38, wherein the acid is acetic acid or hydrochloric acid.

40. The method of claim 39, wherein the acetic acid or hydrochloric acid is about 20 mM, about 30 mM, about 40 mM, about 50 mM, about 60 mM, about 70 mM, about 80 mM, about 90 mM, about 100 mM, about 200 mM, about 300 mM, about 400 mM, about 500 mM, or about 600 mM.

41. The method of claim 40, wherein the acetic acid is about 30 mM.

41. The method of claim 38, further comprising mixing the neutralized pre-treated biological sample with a lysosomal enzyme.

42. The method of claim 41, wherein the neutralized pre-treated biological sample and the lysosomal enzyme mixture is added to a high throughput plate coated with a lysosomal enzyme antigen.

43. The method of claim 42, wherein the lysosomal enzyme antigen is selected from a group consisting of: an α-Gal A antigen, a glucocerebrosidase antigen, an alpha-glucosidase antigen, an alpha-L-iduronidase antigen, a iduronate 2-sulfatase antigen, a sulfamidase antigen, a galactosamine-6-sulfatase antigen, a N-acetylgalactosamine-4 sulfatase antigen, a beta-glucuronidase antigen, a N-acetylglucosamine-1-phosphotransferase antigen, a Niemann-Pick C1 protein antigen, a hexosaminidase A antigen, and a tripeptidly peptidase 1 antigen.

44. The method of claim 43, wherein the lysosomal enzyme antigen is the α-Gal A antigen.

45. The method of any one of claims 42 to 44, wherein an enzyme-conjugated detection antibody or enzyme-conjugated lysosomal enzyme is added to the plate to produce a signal.

46. The method of any one of claims 42 to 44, wherein a detection antibody or lysosomal enzyme which has a ruthenylated label is added to the plate to produce a signal.

47. The method of claim 45 or 46, wherein the detection antibody is an anti-human IgG antibody.

48. The method of claim 45 or 47, further comprising adding a substrate.

49. The method of claim 48, wherein the substrate is converted by the enzyme on the detection antibody, producing a color reaction product.

50. The method of claim 49, wherein the plate is read in a plate reader which detects the color reaction product and outputs optical density (OD) values.

51. The method of claim 50, wherein the OD values represent the total levels of the anti-lysosomal neutralizing antibody.

52. The method of claim 46, wherein the plate is read in a plate reader which detects light emissions by the ruthenylated label and outputs electrochemiluminescent units (ECLu).

53. The method of claim 52, wherein the ECLu represent the total levels of the anti-lysosomal total antibodies.

54. The method of any one of claims 1 to 53, wherein the presence of the anti-lysosomal enzyme antibody is measured in the presence of a circulating lysosomal enzyme in the biological sample.
